(19) United States
(12) Patent Application Publication
(10) Pub. No.: US 2022/0409526 A1
(43) Pub. Date: Dec. 29, 2022

(54) COMPOUNDS AND IMPLANTS FOR TREATING OCULAR DISORDERS

(71) Applicant: Retina Foundation Of The Southwest, Dallas, TX (US)

(72) Inventors: Karl Csaky, Dallas, TX (US); Chandima Bulumulla, Dallas, TX (US); Ruvanthi Kularatne, Dallas, TX (US)

(21) Appl. No.: 17/770,511

(22) PCT Filed: Nov. 6, 2020
(86) PCT No.: PCT/US2020/059518

§ 371 Date: Apr. 20, 2022

Related U.S. Application Data
(60) Provisional application No. 62/932,621, filed on Nov. 8, 2019.

(51) Int. Cl. A61K 9/00; A61P 27/02; A61K 31/223; A61K 31/455

(52) U.S. Cl. CPC A61K 9/0051; A61P 27/02; A61K 31/223; A61K 31/455

(57) ABSTRACT

The present disclosure relates to therapeutic compositions and therapies for use in the treatment of diseases and disorders of the eye. The present disclosure relates to curved, multilayer controlled-release ocular implant devices which include the therapeutic compositions of the present disclosure. The present disclosure related to methods for delivery of the therapeutic agents to the eye and the treatment of diseases and disorders of the eye.

### RELATED APPLICATIONS

The present application claims the benefit of priority under 35 U.S.C. § 119(e) to U.S. Provisional Application No. 62/932,621 filed Nov. 8, 2019, the entire contents of which is incorporated herein by reference in its entirety.

### FIELD OF THE DISCLOSURE

The present disclosure relates to therapeutic compositions and therapies for use in the treatment of diseases and disorders of the eye. The present disclosure relates to curved, multilayer controlled-release ocular implant devices which include the therapeutic compositions of the present disclosure. The present disclosure related to methods for delivery of the therapeutic agents to the eye and the treatment of diseases and disorders of the eye.

### BACKGROUND

Implantable, sustained-release delivery devices can be effective tools in the treatment of many diseases and disorders of the eye, especially in the case of degenerative or persistent conditions. Particularly useful are devices which continuously administers a therapeutic agent to the eye for a prolonged period of time.

However, due to the sensitive nature of the eye and ocular cavity, producing stable, biocompatible ocular implants which provide effective and safe sustained release of therapeutic compositions is difficult. A need therefore exists for improved therapeutic compositions and corresponding implant materials (such as polymers) for delivery of the therapeutic composition.

### SUMMARY

The present disclosure presents therapeutic compositions for use in the treatment of diseases and disorders of the eye. In certain embodiments, the therapeutic compositions include a therapeutic agent. In certain embodiments, the therapeutic agent is an N-acetylcysteine (NAC) alkyl-ester analogue. In certain embodiments, the therapeutic agent is a NAC alkyl-ester analogue according to Formula (I):

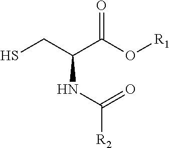

In certain embodiments, R1 is a C1-C5 branched or linear alkyl group including methyl, ethyl, n-propyl, isopropyl, n-butyl, sec-butyl, isobutyl, t-butyl, n-pentyl, or sec-pentyl. In certain embodiments, R1 is a C1-C4 linear alkyl group. In certain embodiments, R2 is a C1-C3 alkyl or a pyridyl group. In certain embodiments, R2 is a C1-C2 alkyl or a pyridyl group. In certain embodiments, R2 is a pyridyl group. In certain embodiments, R2 is a C1-C2 alkyl group including methyl or ethyl. In certain embodiments, R2 is a C1-C3 alkyl group including methyl, ethyl or propyl including n-propyl and iso-propyl. In certain embodiments, R1 is a C1-C5 branched or linear alkyl group, and R2 is a C1-C2 alkyl group or pyridyl group. In certain embodiments, R1 is a C1-C4 linear alkyl group, and R2 is a C1-C2 alkyl group. In certain embodiments, R1 is a C1-C4 linear alkyl group, and R2 is a C1-C3 alkyl group. In certain embodiments, R1 is C1-C4 linear alkyl group; and R2 is a pyridyl group.

In certain embodiments, the therapeutic agent is selected from: an N-acetylcysteine methyl ester (NACME), an N-acetylcysteine ethyl ester (NACEE), an N-acetylcysteine propyl ester (NACPE) including n N-acetylcysteine isopropyl ester, an N-acetylcysteine butyl ester (NACBE), an N-nicotinoylcysteine methyl ester (NNICME), an N-nicotinoylcysteine ethyl ester (NNICEE), or an N-nicotinoylcysteine propyl ester (NNICPE), including N-nicotinoylcysteine isopropyl ester. In certain embodiments, the therapeutic agent is an N-acetylcysteine methyl ester (NACME). In certain embodiments, the therapeutic agent is an N-acetylcysteine ethyl ester (NACEE). In certain embodiments, the therapeutic agent is an N-acetylcysteine propyl ester (NACPE).). In certain embodiments, the therapeutic agent is an N-acetylcysteine isopropyl ester. In certain embodiments, the therapeutic agent is an N-acetylcysteine butyl ester (NACBE). In certain embodiments, the therapeutic agent is an N-nicotinoylcysteine methyl ester (NNICME). In certain embodiments, the therapeutic agent is an N-nicotinoylcysteine ethyl ester (NNICEE). In certain embodiments, the therapeutic agent is an N-nicotinoylcysteine propyl ester (NNICPE). In certain embodiments, the therapeutic agent is an N-nicotinoylcysteine isopropyl ester.

In certain embodiments, the present disclosure presents an ocular implant which includes a biocompatible polymer. In certain embodiments, the ocular implant includes a NAC alkyl-ester analogue of the present disclosure and a biocompatible polymer. In certain embodiments, the ocular implant includes a NAC alkyl-ester analogue of the present disclosure dispersed within a biocompatible polymer.

In certain embodiments, the biocompatible polymer includes an ethylene-vinyl ester copolymer. In certain embodiments, the biocompatible polymer includes an ethylene-vinyl ester copolymer selected from: ethylene-vinyl acetate (EVA), ethylene-vinyl hexanoate (EVH), ethylene-vinyl propionate (EVP), ethylene-vinyl butyrate (EVB), ethylene vinyl pentantoate (EVP), ethylene-vinyl trimethyl acetate (EVTMA), ethylene-vinyl diethyl acetate (EVDEA), ethylene-vinyl 3-methylbutanoate (EVMB), ethylene-vinyl 3-3-dimethylbutanoate (EVDMB), ethylene-vinyl benzoate (EVBZ), or mixtures thereof. In certain embodiments, the biocompatible polymer includes an ethylene-vinyl acetate (EVA) copolymer.

In certain embodiments, the present disclosure presents a multilayer ocular implant which includes a biocompatible polymer of the present disclosure. In certain embodiments, the multilayer ocular implant includes a NAC alkyl-ester analogue of the present disclosure and a biocompatible polymer of the present disclosure. In certain embodiments, the multilayer ocular implant includes a NAC alkyl-ester analogue of the present disclosure dispersed within a biocompatible polymer of the present disclosure.

In certain embodiments, the multilayer ocular implant includes an outer layer and an inner layer. In certain embodiments, the multilayer ocular implant includes an outer layer which includes a first polymer. In certain embodiments, the outer layer includes curvature at both an outer surface and an inner surface. In certain embodiments, the multilayer ocular implant includes an inner layer which includes a second polymer. In certain embodiments, the inner layer includes a biocompatible polymer of the present disclosure and a therapeutic composition of the present disclosure. In certain embodiments, the inner layer includes curvature at both an outer surface and an inner surface. In certain embodiments, the outer layer extends circumferentially beyond the inner layer such that the surface of the circumferential extension of the outer layer is capable of making contact with the sclera of an eye. In certain embodiments, at least one surface of the inner layer is capable of making contact with the sclera of the eye.

In certain embodiments, the outer layer is resistant to diffusion of the therapeutic agent from the inner layer. In certain embodiments, the outer layer is substantially impermeable to diffusion of the therapeutic agent from the inner layer.

In certain embodiments, the first polymer in the outer layer is selected from: polyvinyl acetate, cross-linked poly(vinyl alcohol), cross-linked poly(vinyl butyrate), ethylene ethylacrylate co-polymer, poly(ethyl hexylacrylate), poly(vinyl chloride), poly(vinyl acetals), plasiticized ethylene vinylacetate copolymer, poly(vinyl alcohol), poly(vinyl acetate), ethylene vinylchloride copolymer, poly(vinyl esters), polyvinylbutyrate, polyvinylformal, polyamides, poly(methyl methacrylate), poly(butyl methacrylate), plasticized poly(vinyl chloride), plasticized nylon, plasticized soft nylon, plasticized poly(ethylene terephthalate), natural rubber, polyisoprene, polyisobutylene, polybutadiene, polyethylene, polytetrafluoroethylene, poly(vinylidene chloride), polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer or vinylidene chloride-acrylonitride copolymer.

In certain embodiments, the outer layer and the inner layer are each about 1 mm thick. In certain embodiments, the outer layer or the inner layer includes an agent that blocks lymphatic absorption of the therapeutic agent. In certain embodiments, the inner layer includes a permeability agent that enhances permeability of the therapeutic agent into the eye. In certain embodiments, the outer layer and the inner layer are bound together by a pressure sensitive silicone adhesive.

In certain embodiments, the present disclosure presents methods of treating diseases and disorders of the eye using the therapeutic compositions and implants of the present disclosure. In certain embodiments, the method includes providing a therapeutic composition of the present disclosure or an ocular implant of the present disclosure; and placing the therapeutic composition or the ocular implant into the sub-Tenon's space and in contact with the sclera of the eye of the subject. In certain embodiments, the therapeutic composition or the ocular implant is placed in the posterior of the eye near the macula of the eye. In certain embodiments, an applicator device is used to place the therapeutic composition or the ocular implant into the sub-Tenon's space the eye.

In certain embodiments, the eye disorder is macular degeneration. In certain embodiments, the eye disorder is age-related macular degeneration (AMD).

The present disclosure provides a shaped ocular implant for delivery of drugs to the eye for treatment of diseases and disorders of the eye.

Local ocular implants avoid the shortcomings and complications that can arise from systemic therapies of eye disorders. For instance, oral therapies for the eye fail to provide sustained-release of the drug into the eye. Instead, oral therapies often only result in negligible actual absorption of the drug in the ocular tissues due to low bioavailability of the drug. Ocular drug levels following systemic administration of drugs is usually limited by various blood/ocular barriers (i.e., tight junctions between the endothelial cells of the capillaries). These barriers limit the amounts of drugs entering the eye via systemic circulation. In addition, variable gastrointestinal drug absorption and/or liver metabolism of the medications can lead to dosage-dependent and inter-individual variations in vitreous drug levels. Moreover, adverse side effects have been associated with systemic administration of certain drugs to the eyes.

For instance, systemic treatments of the eye using the immune response modifier cyclosporine A (CsA) have the potential to cause nephrotoxicity or increase the risk of opportunistic infections, among other concerns. This is unfortunate since CsA is a recognized effective active agent for treatment of a wide variety of eye diseases and indications, such as endogenous or anterior uveitis, corneal transplantation, Behçet's disease, vernal or ligneous keratoconjunctivitis, dry eye syndrome, and the like. In addition, rejection of corneal allografts and stem cell grafts occurs in up to 90% of patients when associated with risk factors such as corneal neovascularization. CsA has been identified as a possibly useful drug for reducing the failure rate of such surgical procedures for those patients. Thus, other feasible delivery routes for such drugs that can avoid such drawbacks associated with systemic delivery are in demand.

Apart from implant therapies, other local administration routes for the eye have included topical delivery. Such therapies include ophthalmic drops and topical ointments containing the medicament. Tight junctions between corneal epithelial cells limit the intraocular penetration of eye drops and ointments. Topical delivery to the eye surface via solutions or ointments can in certain cases achieve limited, variable penetration of the anterior chamber of the eye. However, therapeutic levels of the drug are not achieved and sustained in the middle or back portions of the eye. This is a major drawback, as the back (posterior) chamber of the eye is a frequent site of inflammation or otherwise the site of action where, ideally, ocular drug therapy should be targeted for many indications.

Therapeutic agents for the treatment of the eye can be broadly divided into two groups: hydrophilic compounds and lipophilic compounds. Hydrophilic compounds are well established and have a wide range of therapeutic uses due to the ease with which they dissolve in water. However, hydrophilic compounds do not cross lipid barriers easily and, in the eye specifically, lymphatic clearance of compounds in the episclera contributes to the difficulty of maintaining therapeutic levels of the drug as mentioned herein.

Lipophilic compounds do not dissolve easily in an aqueous solution, but due to their chemical nature may easily cross lipid membranes including the blood-neural barrier in the brain or the blood-retinal barrier in the eye. Therefore, lipophilic compounds represent an emerging class of therapeutic drugs that may circumvent difficulties seen in existing drug treatment methodologies. In some embodiments, the lipophilic agents or drugs employed in the implants of the disclosure collect, concentrate, aggregate or otherwise have an increased concentration in retinal tissues. This retinal trapping or sink effect provides for increased efficacy. Such efficacy may be measured by an increase in one or more phenotypic effects, half-life of the drug at a particular retinal or retinal-related location or durational clinically beneficial effect.

In some embodiments retinal trapping results in an increase of drug substance of at least 10%, 20%, 30%, 40%, 50%, 60%, 70%, or 80% or more of drug to the retinal tissue or cells. In some embodiments, the ratio of drug in the retinal tissue, e.g., retinal trap, compared to either surrounding tissue or drug remaining in the implant at any time is 1.5 to 1, 2 to 1, 3 to 1, 4 to 1 or greater than 5 to 1.

Age-related macular degeneration (AMD) is a common disease associated with aging that gradually impairs sharp, central vision. There are two common forms of AMD: dry AMD and wet AMD. About ninety percent of the cases of AMD are the dry form, caused by degeneration and thinning of the tissues of the macula; a region in the center of the retina that allows people to see straight ahead and to discern fine details. Although only about ten percent of people with AMD have the wet form, it poses a much greater threat to vision. With the wet form of the disease, rapidly growing abnormal blood vessels known as choroidal neovascular membranes (CNVM) develop beneath the macula. These vessels leak fluid and blood that destroy light sensing cells, thereby producing blinding scar tissue, with resultant severe loss of central vision. Wet AMD is the leading cause of legal blindness in the United States for people aged sixty-five or more with approximately 25,000 new cases diagnosed each year in the United States. Ideally, treatments of the indication would include inducing an inhibitory effect on the choroidal neovascularization (CNV) associated with AMD. The macula is located at the back of the eye and therefore treatment of CNVM by topical delivery of pharmacological agents to the tissues of the macula tissues is not possible. Intravitreal injections of anti-angiogenic agents, laser photocoagulation, photodynamic therapy, and surgical removal are currently used to treat CNVM. Unfortunately, the recurrence rate using such methods exceeds 50-90% in some cases. In most cases indefinite treatment is required.

As an approach for circumventing the barriers encountered by local topical delivery, one local therapy route for the eye has involved direct intravitreal injection of a treatment drug through the sclera (i.e., the spherical, collagen-rich outer covering of the eye). However, the intravitreal injection delivery route tends to result in a short half-life and rapid clearance without sustained release capability being attained. Consequently, weekly to monthly injections are frequently required to maintain therapeutic ocular drug levels. This is not practical for many patients.

Given these drawbacks, the use of implant devices placed in or adjacent to the eye tissues to deliver therapeutic drugs thereto should offer a great many advantages and opportunities over the rival therapy routes. Despite the variety of ocular implant devices which have been described and used in the past, the full potential of the therapy route has not been realized. Among other things, prior ocular implant devices deliver the drug to the eye tissues via a single mode of administration for a given treatment, such as via slow constant rate infusion at low dosage. However, in many different clinical situations, such as with CNVM in AMD, this mode of drug administration might be a sub-optimal ocular therapy regimen.

Another problem exists with previous ocular implants, from a construction standpoint, insofar as preparation techniques thereof have relied on covering the drug pellet or core with a permeable polymer by multi-wet coating and drying approaches. Such wet coating approaches can raise product quality control issues such as an increased risk of delamination of the thinly applied coatings during subsequent dippings, as well as thickness variability of the polymer around the drug pellets obtained during hardening. Additionally, increased production costs and time from higher rejection rates and labor and an increased potential for device contamination from additional handling are known problems with present implant technology.

Accordingly, certain aspects of the present disclosure provide local treatment of a variety of eye diseases. Other aspects of the present disclosure also provide a method for the delivery of pharmaceuticals to the eye to effectively treat eye disease, while reducing or eliminating the systemic side effects of these drugs. Certain aspects of the present disclosure also provide shaped sustained-release ocular implants for administration of therapeutic agents to the eye for prolonged periods of time. Additionally, certain aspects of the present disclosure provide approaches to alter the areas of the eye that are affected by diffusion of drugs from sustained-release ocular implants. Certain aspects of the present disclosure also provide methods for making shaped ocular implants with reduced product variability.

Other aspects of the present disclosure also provide methods for making shaped ocular implants well-suited for ocular treatment trials using animal models. Other advantages and benefits of aspects of the present disclosure will be apparent from consideration of the present specification.

In these and other ways described below, the implants of the present disclosure offer a myriad of advantages, improvements, benefits, and therapeutic opportunities. The implants of the present disclosure are highly versatile and can be tailored to enhance the delivery regimen both in terms of administration mode(s) and type(s) of drugs delivered. The implants of this disclosure permit continuous release of therapeutic agents into the eye over a specified period of time, which can be weeks, months, or even years as desired. As another advantage, the implant systems of this disclosure require intervention only for initiation and termination of the therapy (i.e., removal of the implant). Patient compliance issues during a regimen are eliminated. The time-dependent delivery of one or more drugs to the eye by this disclosure makes it possible to maximize the pharmacological and physiological effects of the eye treatment. The implants of the present disclosure have human and veterinary applicability.

In one aspect of the present disclosure, there is provided a method for forming a molded two-layer ocular implant, the implant including a therapeutic agent for treatment or prevention of a disorder of the eye, the method including: a) dispensing a polymer into a curved depression on a mold body to form a polymer layer having a curved external surface in contact with the bottom of the curved depression and further including an exposed upper surface; b) generating a curvature in the exposed upper surface of the polymer layer, thereby forming a curved polymer layer interface surface; c) curing the polymer layer, thereby providing a hardened curved polymer layer interface surface; d) dispensing a silicone adhesive including the therapeutic agent dispersed therein onto the hardened interface surface to provide a silicone layer with an exposed surface; e) generating a curvature in the exposed surface of the silicone layer thereby forming a curved eye-contacting surface; and f) curing the silicone layer such that the first layer and second layer are fixed to each other, thereby forming the molded two-layer ocular implant.

Another aspect of the present disclosure is a method for forming a molded two-layer ocular implant, the implant including a therapeutic agent for treatment or prevention of a disorder of the eye, the method including: a) dispensing a polymer into a curved depression on a first mold body to form a polymer layer having a curved external surface in contact with the bottom of the curved depression and further including an exposed upper polymer surface; b) generating a curvature in the exposed upper surface of the polymer layer, thereby forming a curved polymer layer interface surface; c) curing the polymer layer to produce a cured polymer layer, d) dispensing a silicone adhesive including the therapeutic agent dispersed therein into second curved depression on a second mold body to provide a silicone layer with a curved silicone layer interface surface in contact with the bottom of the curved depression and further including an exposed upper silicone surface; e) generating a curvature in the exposed silicone surface, thereby forming a curved eye-contacting surface; f) curing the silicone layer to produce a cured silicone layer; and g) joining the cured polymer layer to the cured silicone layer by attachment of the polymer layer interface surface to the silicone layer interface surface with biocompatible adhesive. In certain embodiments, the adhesive is pressure sensitive. In certain embodiments, the pressure sensitive adhesive may include any of those from DOW CORNING® such as BIO-PSA 7-4302 or other such adhesives from the DOW CORNING® catalog, the contents of which are incorporated herein by reference in their entirety.

In certain embodiments, the implant is circular or oval-shaped.

In certain embodiments, steps b) and e) are performed using an impression body with a curved protrusion for generating the curvature in the exposed surface of the polymer layer and the exposed surface of the silicone layer.

In certain embodiments, step b) is performed using a first impression body including a first curved protrusion for generating the curvature in the exposed surface of the polymer layer and step e) is performed using a second impression body including a second curved protrusion for generating the curvature in the exposed surface of the silicone layer, wherein the curvature dimensions of the first and second curved protrusions are different.

In certain embodiments, the polymer layer is resistant to diffusion of the therapeutic agent from the silicone layer.

In certain embodiments, the polymer layer is substantially impermeable to diffusion of the therapeutic agent from the silicone layer.

In certain embodiments, the polymer is polyvinyl acetate, cross-linked poly(vinyl alcohol), cross-linked poly(vinyl butyrate), ethylene ethylacrylate co-polymer, poly(ethyl hexylacrylate), poly(vinyl chloride), poly(vinyl acetals), plasiticized ethylene vinylacetate copolymer, poly(vinyl alcohol), poly(vinyl acetate), ethylene vinylchloride copolymer, poly(vinyl esters), polyvinylbutyrate, polyvinylformal, polyamides, poly(methyl methacrylate), poly(butyl methacrylate), plasticized poly(vinyl chloride), plasticized nylon, plasticized soft nylon, plasticized poly(ethylene terephthalate), natural rubber, polyisoprene, polyisobutylene, polybutadiene, polyethylene, polytetrafluoroethylene, poly(vinylidene chloride), polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer or vinylidene chloride-acrylonitride copolymer.

In certain embodiments, the polymer layer and the silicone layer are each about 1 mm thick.

In certain embodiments, the polymer layer and/or the silicone layer further include an agent that blocks lymphatic absorption of the therapeutic agent.

In certain embodiments, the silicone layer further includes an ophthalmic permeation agent that increases ocular permeability of the therapeutic agent into the eye.

In certain embodiments, the ophthalmic permeation agent is methylsulfonylmethane.

In certain embodiments, the radius of curvature of the curved eye-contacting surface of the silicone layer ranges from between about 5 mm to about 6 mm.

In certain embodiments, the resulting molded implant is circular with a diameter ranging between about 1 mm and 8 mm.

In certain embodiments, the resulting molded implant is circular with a diameter ranging between about 1 mm and 3 mm.

In certain embodiments, the therapeutic agent is a nuclear factor (erythroid-derived 2)-like 2 enhancer (Nrf2 regulator).

In certain embodiments, the Nrf2 regulator is sulforaphane.

In certain embodiments, the therapeutic agent is selected from the group consisting of fumagillin analogs, minocycline, fluoroquinolone, cephalosporin antibiotics, herbimycon A, tetracycline, chlortetracycline, bacitracin, neomycin, polymyxin, gramicidin, oxytetracycline, chloramphenicol, gentamicin, erythromycin, antibacterial agents, sulfonamides, sulfacetamide, sulfamethizole, sulfoxazole, nitrofurazone, sodium propionate, antiviral agents, idoxuridine, famvir, trisodium phosphonoformate, trifluorothymidine, acyclovir, ganciclovir, DDI, AZT, protease and integrase inhibitors, anti-glaucoma agents, beta blockers, timolol, betaxolol, atenolol, prostaglandin analogues, hypotensive lipids, carbonic anhydrase inhibitors, antiallergenic agents, antazoline, methapyriline, chlorpheniramine, pyrilamine, prophenpyridamine, anti-inflammatory agents, hydrocortisone, leflunomide, dexamethasone phosphate, fluocinolone acetonide, medrysone, methylprednisolone, prednisolone phosphate, prednisolone acetate, fluoromethalone, betamethasone, triamcinolone acetonide, adrenalcortical steroids and their synthetic analogues, 6-mannose phosphate, antifungal agents, fluconazole, amphotericin B, liposomal amphotericin B, voriconazole, imidazole-based antifungals, tiazole antifungals, echinocandin-like lipopeptide antibiotics, lipid formulations of antifungals, polycations, polyanions, suramine, protamine, decongestants, phenylephrine, naphazoline, tetrahydrazoline, anti-angiogenesis compounds including those that can be potential anti-choroidal neovascularization agents, 2-methoxyestradiol and its analogues, 2-propynl-estradiol, 2-propenyl-estradiol, 2-ethoxy-6-oxime-estradiol, 2-hydroxyestrone, 4-methoxyestradiol, VEGF antagonists, VEGF antibodies and VEGF antisense compounds, angiostatic steroids, anecortave acetate and its analogues, 17-ethynylestradiol, norethynodrel, medroxyprogesterone, mestranol, androgens with angiostatic activity, ethisterone, thymidine kinase inhibitors, adrenocortical steroids and their synthetic analogues, fluocinolone acetonide, triamcinolone acetonide, immunological response modifying agents, cyclosporineA, Prograf (tacrolimus), macrolide immunosuppressants, mycophenolate mofetil, rapamycin, muramyl dipeptide, vaccines, anti-cancer agents, 5-fluorouracil, platinum coordination complexes, cisplatin, carboplatin, adriamycin, antimetabolites, methotrexate, anthracycline antibiotics, antimitotic drugs, paclitaxel, docetaxel, epipdophylltoxins, etoposide, nitrosoureas, carmustine, alkylating agents, cyclophosphamide, arsenic trioxide, anastrozole, tamoxifen citrate, triptorelin pamoate, gemtuzumab ozogamicin, irinotecan hydrochloride, leuprolide acetate, bexarotene, exemestrane, epirubicin hydrochloride, ondansetron, temozolomide, topoteanhydrochloride, tamoxifen citrate, irinotecan hydrochloride, trastuzumab, valrubicin, gemcitabine HCl, goserelin acetate, capecitabine, aldesleukin, rituximab, oprelvekin, interferon alfa-2a, letrozole, toremifene citrate, mitoxantrone hydrochloride, irinotecan HeL, topotecan HCl, etoposide phosphate, amifostine, antisense agents, antimycotic agents, miotic and anticholinesterase agents, pilocarpine, eserine salicylate, carbachol, diisopropyl fluorophosphate, phospholine iodine, demecarium bromide, mydriatic agents such as atropine sulfate, cyclopentane, homatropine, scopolamine, tropicamide, eucatropine, hydroxyamphetamine, differentiation modulator agents, sympathomimetic agents epinephrine, anesthetic agents, lidocaine, benzodiazepam, vasoconstrictive agents, vasodilatory agents, polypeptides, protein agents, angiostatin, endostatin, matrix metalloproteinase inhibitors, platelet factor 4, interferon-gamma, insulin, growth hormones, insulin related growth factor, heat shock proteins, humanized antiIL2 receptor mAb (Daclizumab), etanercept, mono and polyclonal antibodies, cytokines, antibodies to cytokines, neuroprotective agents such as calcium channel antagonists including nimodipine and diltiazem, neuroimmunophilin ligands, neurotropins, memantine, NMDA antagonists, acetylcholinesterase inhibitors, estradiol and analogues, vitamin B12 analogues, alpha-tocopherol, NOS inhibitors, antioxidants, glutathione, superoxide dismutase, cobalt, copper, neurotrophic receptors, Akt kinase, growth factors, nicotinamide (vitamin B3), alpha-tocopherol (vitamin E), succinic acid, dihydroxylipoic acid, fusidic acid, cell transport/mobility impending agents, colchicine, vincristine, cytochalasin B, carbonic anhydrase inhibitor agents, integrin antagonists and lubricating agents.

In certain embodiments, the therapeutic agent is a lipophilic agent. In certain embodiments, the lipophilic therapeutic agent is selected from the group consisting of Idebenone, rapamycin, 2-cyano-3,12 dioxooleana-1,9 dien-28-imidazolide (CDDO-Im), 2-cyano-3,12-dioxooleana-1,9(11)-dien-28-oic acid-ethyl amide (CDDO-ethyl amide), and 2-cyano-3,12-dioxooleana-1,9(11)-dien-28-oic acid trifluoroethyl amide (CDDO-TFEA).

In certain embodiments, the polymer layer and/or the silicone layer further include a nutraceutical oil.

In certain embodiments, the nutraceutical oil is omega-3 fish oil.

In certain embodiments, the silicone layer further includes an excipient that improves the release of drug.

In certain embodiments, the excipient is selected from one or more of isopropyl myristate, levomenthol, propylene and tetraglycol.

Another aspect of the disclosure is a two-layer implant formed by the methods described herein. The implant of certain embodiments may be used for implantation into the sub-Tenon's space of a human. The implant of other embodiments may be used for implantation into the sub-Tenon's space of a rodent.

Another aspect of the disclosure is a molded two-layer ocular implant including a therapeutic agent for treatment or prevention of a disorder of the eye, the implant including: a first hardened layer including a polymer, the first hardened layer including curvature at both surfaces; and a second hardened layer including a silicone adhesive and the therapeutic agent, the second hardened layer and including curvature at both surfaces.

In certain embodiments, the curvature of one surface of the first hardened layer and the curvature of one surface of the second layer are both formed using an impression body with a curved protrusion.

In certain embodiments, the first and second hardened layers are defined as follows: the curvature of a first surface of the first hardened layer is formed by dispensing the polymer into a mold body; the curvature of a second surface of the first hardened layer is formed by a first curved protrusion on a first impression body; the curvature of a first surface of the second hardened layer is formed by dispensing the silicone adhesive onto the curvature of the second surface of the first hardened layer; and the curvature of a second surface of the second hardened layer is formed by a second curved protrusion on a second impression body.

In certain embodiments, the first hardened layer is resistant to diffusion of the therapeutic agent from the second hardened layer.

In certain embodiments, the first hardened layer is substantially impermeable to diffusion of the therapeutic agent from the second hardened layer.

Another aspect of the present disclosure is a mold assembly for forming a two-layer ocular implant, the mold assembly including: a mold body including a contact surface with a curved depression formed therein for forming a first curved surface of a polymer layer of the implant; and an impression body including a curved protrusion for forming curvature at a second surface of the polymer layer and for forming curvature in a surface of a silicone adhesive layer of the implant.

In certain embodiments, the curved protrusion is for forming curvature in only the second surface of the polymer layer of the implant and the mold assembly further includes a second impression body including a second curved protrusion for forming the curvature in the surface of the silicone adhesive layer of the implant.

In certain embodiments, the impression body is mounted on a support frame configured to allow vertical movement of the impression body and the support frame while the mold body remains stationary and the support frame further includes a means for locking of the position of the impression body.

In certain embodiments, the mold assembly further includes a means for controlling the thickness of the polymer layer and the silicone adhesive layer formed by the mold body and impression body.

In certain embodiments, the mold body is cylindrical and dimensioned for insertion in a centrifuge tube.

In certain embodiments, the surfaces of the depression and the protrusion are coated with a non-stick material to facilitate removal of the implant from the mold body.

In certain embodiments, the non-stick material is Teflon® or aluminum.

Another aspect of the present disclosure is a method for determining the effectiveness of the implant as described herein for treatment or prevention of macular degeneration in a rodent, the method including: a) placing the implant as described herein in the sub-Tenon's space of the eye of the rodent, wherein the rodent is fed with high-fat chow supplemented with hydroquinone; and b) monitoring the release of the drug over time by examining the eye of the rodent with histology, electroretinography or changes in gene expression the retinal pigment epithelium or photoreceptors, thereby indicating the effectiveness of the implant against macular degeneration.

Another aspect of the present disclosure is a method for evaluating the effectiveness of the implant as described herein for treatment or prevention of macular degeneration in a human, the method including: a) placing the implant as described herein into the sub-Tenon's space of the eye of the human; and b) examining the eye of the human using a technique selected from the group consisting of: 2 color (blue, red) microperimetry, low luminance visual acuity, multi-focal electroretinography, dynamic perimetry, color vision assessment, photo-stress testing and static perimetry, thereby evaluating the effectiveness of the implant against macular degeneration.

Another aspect of the present disclosure is a kit for preparing a molded two-layer composite ocular implant including a therapeutic agent for treatment or prevention of a disorder of the eye, the kit including: a) a mold assembly for molding the implant; b) a silicone adhesive including a therapeutic agent for forming a first layer; and c) a polymer for forming a second layer.

In certain embodiments, the mold assembly of the kit is the mold assembly described herein which includes a single impression body. In other embodiments, the mold assembly of the kit is the mold assembly which includes two impression bodies.

In certain embodiments, the kit further includes instructions for making a molded two-layer silicon composite ocular implant by sequential layering of the polymer and the silicone adhesive including the therapeutic agent.

### BRIEF DESCRIPTION OF THE FIGURES

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIG. 1A presents a perspective view of implant 10 according to one embodiment of the disclosure with curved lines 12 and 14 showing the curvature of the upper surface of the implant. FIG. 1B presents atop view of implant 10.

FIG. 2 presents a cross sectional side view of implant 10 taken along line 3′-3′ of FIG. 1B (along dotted line 14) showing the lower layer 16 and upper layer 18 of the implant with drug particles 20 dispersed in the lower layer 16. Features of the implant are omitted for clarity.

FIG. 3 presents a schematic side slice view showing selected anatomy of an eye E with the placement of a perspective view of implant 10 in the sub-Tenon's space E0. Other structures of the eye E are shown for context.

FIG. 4 presents a magnified view of the rectangular inset 5′ of FIG. 3 showing a perspective view of implant 10. Also shown are additional layers of structures and tissues within the eye and diffusion of a drug 20 to the sclera E3 and the choroid E4.

FIG. 5 presents an exemplary synthesis scheme for NAC alkyl-ester analogues of the present disclosure, as well as a schematic representation of increasing lipophilicity from NAC to NACBE.

FIG. 6A presents the results of a dose responsive XTT assay for HQ. ARPE-19 cells were exposed to 100-1000 μM HQ for 16 hours. FIG. 6B presents the results of a time dependent XTT assay for NAC and NAC alkyl-ester analogues with a 16-hour exposure to 500 μM HQ. ARPE-19 cells were pretreated with NAC and NAC alkyl-ester analogues for 2, 24 and 48 hours followed by the exposure to 500 μM HQ for 16 hours. FIG. 6C presents the results of a dose dependent XTT assay for NAC and NACBE. ARPE-19 cells were pretreated with NAC and NACBE at 0.001-1.0 mM for 24 hours followed by exposure to 500 μM HQ for 16 hours.

FIG. 7 presents confocal images of ARPE-19 cells with ZO-1 staining expressing cellular junctions. ARPE-19 cells were pretreated with 1 mM NAC and NACBE followed by 2-hour exposure to 500 μM HQ.

FIG. 8 presents the results from an HPLC chromatograms of ARPE-19 cells with and without treatment with 1 mM NAC and NACBE.

FIG. 9 presents the results from a GSH assay for NAC, NAC ester derivatives, NACA and GSH-EE. ARPE-19 cells were exposed to 1 mM drug concentration for 24 hours before measuring cytoplasmic GSH levels.

FIG. 10A presents an exemplary synthesis scheme for dansyl tagged NAC alkyl-ester analogues of the present disclosure. FIG. 10B presents UV-Vis absorbance spectra for Dan-NACME, Dan-NACEE, Dan-NACPE and Dan-NACBE in PBS. FIG. 10C presents fluorescence spectra of Dan-NACME, Dan-NACEE, Dan-NACPE and Dan-NACBE in PBS.

FIG. 11 presents confocal images of ARPE-19 cells exposed to NACBE, Dan-NACME, Dan-NACEE, Dan-NACPE and Dan-NACBE at 1 mM for 1 and 24 hours.

FIG. 12A presents JC-1 assay results for ARPE-19 cells exposed to 25, 50 and 100 μM HQ at 1, 2, 4, 6, 8 and 16 hours. FIG. 12B presents JC-1 assay for ARPE-19 cells pretreated with 1 mM NAC, NAC ester derivatives, NACA, GSH-EE and 1 μM MitoQ for 1 and 24 hours before exposing to 50 μM HQ for 4 hours.

FIG. 13 presents confocal images of ARPE-19 cells treated with 10 μM JC-1, 10 μM JC-1+50 μM HQ, 10 μM JC-1+50 μM HQ pretreated with 1 mM NAC and JC-1+50 μM HQ pretreated with 1 mM NACBE. The cells were pretreated with NAC alkyl-ester analogues of the present disclosure for 24 hours before exposing to 50 μM HQ for 4 hours.

FIG. 14 presents mitochondrial GSH assay results after treating ARPE-19 cells with 1 mM NAC and NACBE for 24 hours.

FIG. 15 presents CellTiter-Glo assay results for ARPE-19 cells for 500 μM HQ, 1 mM NAC+500 μM HQ and 1 mM NACBE+500 μM HQ for 3, 6 and 8 hours.

FIG. 16 presents relative amplification results of a large band of mitochondrial DNA from ARPE-19 cells treated with 500 μM HQ, 500 μM HQ pretreated with 1 mM NAC, and 500 NM HQ pretreated with 1 mM NACBE.

### DETAILED DESCRIPTION

### I. Therapeutic Agents

### Overview

Therapeutic agents for the treatment of the eye can be broadly divided into two groups: hydrophilic compounds and lipophilic compounds. Hydrophilic compounds are well established and have a wide range of therapeutic uses due to the ease with which they dissolve in water. However, hydrophilic compounds do not cross lipid barriers easily and, in the eye specifically, lymphatic clearance of compounds in the episclera contributes to the difficulty of maintaining therapeutic levels of the drug as mentioned herein.

Lipophilic compounds do not dissolve easily in an aqueous solution, but due to their chemical nature may easily cross lipid membranes including the blood-neural barrier in the brain or the blood-retinal barrier in the eye. Therefore, lipophilic compounds represent an emerging class of therapeutic drugs that may circumvent difficulties seen in existing drug treatment methodologies. In some embodiments, the lipophilic agents or drugs employed in the implants of the disclosure collect, concentrate, aggregate or otherwise have an increased concentration in retinal tissues. This retinal trapping or sink effect provides for increased efficacy. Such efficacy may be measured by an increase in one or more phenotypic effects, half-life of the drug at a particular retinal or retinal-related location or durational clinically beneficial effect.

In some embodiments retinal trapping results in an increase of drug substance of at least 10%, 20%, 30%, 40%, 50%, 60%, 70%, or 80% or more of drug to the retinal tissue or cells. In some embodiments, the ratio of drug in the retinal tissue, e.g., retinal trap, compared to either surrounding tissue or drug remaining in the implant at any time is 1.5 to 1, 2 to 1, 3 to 1, 4 to 1 or greater than 5 to 1.

A number of different therapeutic agents can be delivered to the eye by the ocular implant of the present disclosure. Such therapeutic agents include, but are not limited to: antibiotic agents such as fumagillin analogs, minocycline, fluoroquinolone, cephalosporin antibiotics, herbimycon A, tetracycline, chlortetracycline, bacitracin, neomycin, polymyxin, gramicidin, oxytetracycline, chloramphenicol, gentamicin and erythromycin; antibacterial agents such as sulfonamides, sulfacetamide, sulfamethizole, sulfoxazole, nitrofurazone, and sodium propionate; antiviral agents such as idoxuridine, famvir, trisodium phosphonoformate, trifluorothymidine, acyclovir, ganciclovir, DDI and AZT, protease and integrase inhibitors; anti-glaucoma agents such as beta blockers (timolol, betaxolol, atenolol), prostaglandin analogues, hypotensive lipids, and carbonic anhydrase inhibitors; antiallergenic agents such as antazoline, methapyriline, chlorpheniramine, pyrilamine and prophenpyridamine; anti-inflammatory agents such as hydrocortisone, leflunomide, dexamethasone phosphate, fluocinolone acetonide, medrysone, methylprednisolone, prednisolone phosphate, prednisolone acetate, fluoromethalone, betamethasone, triamcinolone acetonide, adrenalcortical steroids and their synthetic analogues, and 6-mannose phosphate; antifungal agents such as fluconazole, amphotericin B, liposomal amphotericin B, voriconazole, imidazole-based antifungals, tiazole antifungals, echinocandin-like lipopeptide antibiotics, lipid formulations of antifungals; polycations and polyanions such as suramine and protamine; decongestants such as phenylephrine, naphazoline, and tetrahydrazoline; anti-angiogenesis compounds including those that can be potential anti-choroidal neovascularization agents such as 2-methoxyestradiol and its analogues (e.g., 2-propynl-estradiol, 2-propenyl-estradiol, 2-ethoxy-6-oxime-estradiol, 2-hydroxyestrone, 4-methoxyestradiol), VEGF antagonists such as VEGF antibodies and VEGF antisense, angiostatic steroids (e.g., anecortave acetate and its analogues, 17-ethynylestradiol, norethynodrel, medroxyprogesterone, mestranol, androgens with angiostatic activity such as ethisterone), thymidine kinase inhibitors; adrenocortical steroids and their synthetic analogues including fluocinolone acetonide and triamcinolone acetonide and all angiostatic steroids; immunological response modifying agents such as cyclosporineA, Prograf (tacrolimus), macrolide immunosuppressants, mycophenolate mofetil, rapamycin, and muramyl dipeptide, and vaccines; anti-cancer agents such as 5-fluorouracil, platinum coordination complexes such as cisplatin and carboplatin, adriamycin, antimetabolites such as methotrexate, anthracycline antibiotics, antimitotic drugs such as paclitaxel and docetaxel, epipdophylltoxins such as etoposide, nitrosoureas including carmustine, alkylating agents including cyclophosphamide; arsenic trioxide; anastrozole; tamoxifen citrate; triptorelin pamoate; gemtuzumab ozogamicin; irinotecan hydrochloride; leuprolide acetate; bexarotene; exemestrane; epirubicin hydrochloride; ondansetron; temozolomide; topoteanhydrochloride; tamoxifen citrate; irinotecan hydrochloride; trastuzumab; valrubicin; gemcitabine HCL; goserelin acetate; capecitabine; aldesleukin; rituximab; oprelvekin; interferon alfa-2a; letrozole; toremifene citrate; mitoxantrone hydrochloride; irinotecan HeL; topotecan HCL; etoposide phosphate; gemcitabine HCL; and amifostine; antisense agents; antimycotic agents; miotic and anticholinesterase agents such as pilocarpine, eserine salicylate, carbachol, diisopropyl fluorophosphate, phospholine iodine, and demecarium bromide; mydriatic agents such as atropine sulfate, cyclopentane, homatropine, scopolamine, tropicamide, eucatropine, and hydroxyamphetamine; differentiation modulator agents; sympathomimetic agents such as epinephrine; anesthetic agents such as lidocaine and benzodiazepam; vasoconstrictive agents; vasodilatory agents; polypeptides and protein agents such as angiostatin, endostatin, matrix metalloproteinase inhibitors, platelet factor 4, interferon-gamma, insulin, growth hormones, insulin related growth factor, heat shock proteins, humanized antiIL2 receptor mAb (Daclizumab), etanercept, mono and polyclonal antibodies, cytokines, antibody to cytokines; neuroprotective agents such as calcium channel antagonists including nimodipine and diltiazem, neuroimmunophilin ligands, neurotropins, memantine and other NMDA antagonists, acetylcholinesterase inhibitors, estradiol and analogues, vitamin B12 analogues, alpha-tocopherol, NOS inhibitors, antioxidants (e.g. glutathione, superoxide dismutase), metals like cobalt and copper, neurotrophic receptors (Akt kinase), growth factors, nicotinamide (vitamin B3), alpha-tocopherol (vitamin E), succinic acid, dihydroxylipoic acid, fusidic acid; cell transport/mobility impending agents such as colchicine, vincristine, cytochalasin B; carbonic anhydrase inhibitor agents; integrin antagonists; lipophilic agents such as Idebenone, rapamycin, 2-cyano-3,12 dioxooleana-1,9 dien-28-imidazolide (CDDO-Im), 2-cyano-3,12-dioxooleana-1,9(11)-dien-28-oic acid-ethyl amide (CDDO-ethyl amide), and 2-cyano-3,12-dioxooleana-1,9(11)-dien-28-oic acid trifluoroethyl amide (CDDO-TFEA); and lubricating agents. Any of these therapeutic agents may be included in the ocular implant either singly or in combinations thereof.

In certain embodiments, the therapeutic agent is a nuclear factor (erythroid-derived 2)-like 2 enhancer (Nrf2 regulator). In certain embodiments, the Nrf2 regulator is sulforaphane.

Other drugs that could be delivered by the ocular implant include, for example, thalidomide. Reference can be made to Remington's Pharmaceutical Sciences, Mack Publishing Press, Easton, Pa., U.S.A., to identify other possible therapeutic agents for the eye.

Any pharmaceutically acceptable form of the agents can be used, such as the free base form or a pharmaceutically acceptable salt or ester thereof. In this particular embodiment, the dosage of the therapeutic agent provided by the implant is in the range of 1-100 mg, which is an appropriate dosage for a drug such as sulforaphane which is used in the treatment of macular degeneration.

In accordance with the present disclosure, the therapeutic agent or component of the implant may include, consists essentially of, or consists of, a lipophilic agent. Such lipophilic agents may be small molecules. Lipophilic agents may be released from the implant by diffusion, erosion, dissolution or osmosis. The drug release sustaining component may include one or more biodegradable polymers or one or more non-biodegradable polymers.

In one embodiment, the intraocular implants include a lipophilic agent. Lipophilic agents or other agent which may be employed in the implants of the present disclosure include those taught in US Patent Publication, US20140031408, the contents of which are incorporated herein by reference in its entirety.

In another embodiment, intraocular implants include a therapeutic agent or component that includes a lipophilic agent.

### NAC Alkyl-Ester Analogue

The present disclosure presents therapeutic compositions for use in the treatment of diseases and disorders of the eye. In certain embodiments, the therapeutic compositions include a therapeutic agent. In certain embodiments, the therapeutic agent is an N-acetylcysteine (NAC) alkyl-ester analogue. In certain embodiments, the therapeutic agent is a NAC alkyl-ester analogue according to Formula (I):

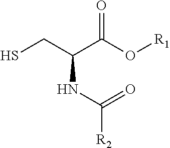

In certain embodiments, R1 is a C1-C5 branched or linear alkyl group. In certain embodiments, R1 is a C1-C4 linear alkyl group. In certain embodiments, R2 is a C1-C3 alkyl group or pyridyl group. In certain embodiments, R2 is a C1-C2 alkyl group or pyridyl group. In certain embodiments, R2 is a pyridyl group. In certain embodiments, R2 is a C1-C3 alkyl group including methyl, ethyl, n-propyl or isopropyl. In certain embodiments, R2 is a C1-C2 alkyl group. In certain embodiments, R1 is a C1-C5 branched or linear alkyl group, and R2 is a C1-C2 alkyl group or pyridyl group. In certain embodiments, R1 is a C1-C4 linear alkyl group, and R2 is a C1-C3 alkyl group. In certain embodiments, R1 is a C1-C4 linear alkyl group, and R2 is a C1-C2 alkyl group. In certain embodiments, R1 is C1-C4 linear alkyl group; and R2 is a pyridyl group.

In certain embodiments, the therapeutic agent is selected from: an N-acetylcysteine methyl ester (NACME), an N-acetylcysteine ethyl ester (NACEE), an N-acetylcysteine propyl ester (NACPE), an N-acetylcysteine butyl ester (NACBE), an N-nicotinoylcysteine methyl ester (NNICME), or an N-nicotinoylcysteine ethyl ester (NNICEE). In certain embodiments, the therapeutic agent is an N-acetylcysteine methyl ester (NACME). In certain embodiments, the therapeutic agent is an N-acetylcysteine ethyl ester (NACEE). In certain embodiments, the therapeutic agent is an N-acetylcysteine propyl ester (NACPE). In certain embodiments, the therapeutic agent is an N-acetylcysteine propyl ester (NACPE). In certain embodiments, the therapeutic agent is an N-acetylcysteine isopropyl ester (NACPE). In certain embodiments, the therapeutic agent is an N-acetylcysteine butyl ester (NACBE). In certain embodiments, the therapeutic agent is an N-nicotinoylcysteine methyl ester (NNICME). In certain embodiments, the therapeutic agent is an N-nicotinoylcysteine ethyl ester (NNICEE). In certain embodiments, the therapeutic agent is an N-nicotinoylcysteine propyl ester (NNICPE). In certain embodiments, the therapeutic agent is an N-nicotinoylcysteine isopropyl ester (NNICPE).

In certain embodiments, the present disclosure presents an ocular implant which includes a biocompatible polymer. In certain embodiments, the ocular implant includes a NAC alkyl-ester analogue of the present disclosure and a biocompatible polymer. In certain embodiments, the ocular implant includes a NAC alkyl-ester analogue of the present disclosure dispersed within a biocompatible polymer.

Without being bound by theory, upon cell uptake, NAC alkyl-ester analogues will undergo de-esterification via endogenous esterases to produce NAC, which will then be converted to cysteine through the activity of amidases. The produced cysteine will then participate in GSH synthesis, thereby increasing the availability of GSH to the cell. GSH, a ubiquitous intracellular antioxidant, then protects cells against oxidative injury.

### II. Ocular Implants

The present disclosure provides a molded composite ocular implant including a therapeutic agent of the present disclosure, including therapeutic agents for treatment or prevention of a disorder of the eye. Also provided are methods of making the composite ocular implant and using the implant for treatment of various diseases or disorders of the eye, including tests of the implant with experimental animals such as rodents. In certain embodiments, the implant provides sustained release of the therapeutic agent during the treatment or prevention of the disorder of the eye. A sustained release implant configuration is particularly well-suited for placement in the sub-Tenon's space (also known as the bulbar sheath) but is not limited thereto and could be installed on or in other eye regions where convenient and useful.

The present disclosure provides a shaped ocular implant for delivery of drugs to the eye for treatment of diseases and disorders of the eye.

Local ocular implants avoid the shortcomings and complications that can arise from systemic therapies of eye disorders. For instance, oral therapies for the eye fail to provide sustained-release of the drug into the eye. Instead, oral therapies often only result in negligible actual absorption of the drug in the ocular tissues due to low bioavailability of the drug. Ocular drug levels following systemic administration of drugs is usually limited by various blood/ocular barriers (i.e., tight junctions between the endothelial cells of the capillaries). These barriers limit the amounts of drugs entering the eye via systemic circulation. In addition, variable gastrointestinal drug absorption and/or liver metabolism of the medications can lead to dosage-dependent and inter-individual variations in vitreous drug levels. Moreover, adverse side effects have been associated with systemic administration of certain drugs to the eyes.

For instance, systemic treatments of the eye using the immune response modifier cyclosporine A (CsA) have the potential to cause nephrotoxicity or increase the risk of opportunistic infections, among other concerns. This is unfortunate since CsA is a recognized effective active agent for treatment of a wide variety of eye diseases and indications, such as endogenous or anterior uveitis, corneal transplantation, Behçet's disease, vernal or ligneous keratoconjunctivitis, dry eye syndrome, and the like. In addition, rejection of corneal allografts and stem cell grafts occurs in up to 90% of patients when associated with risk factors such as corneal neovascularization. CsA has been identified as a possibly useful drug for reducing the failure rate of such surgical procedures for those patients. Thus, other feasible delivery routes for such drugs that can avoid such drawbacks associated with systemic delivery are in demand.

Apart from implant therapies, other local administration routes for the eye have included topical delivery. Such therapies include ophthalmic drops and topical ointments containing the medicament. Tight junctions between corneal epithelial cells limit the intraocular penetration of eye drops and ointments. Topical delivery to the eye surface via solutions or ointments can in certain cases achieve limited, variable penetration of the anterior chamber of the eye. However, therapeutic levels of the drug are not achieved and sustained in the middle or back portions of the eye. This is a major drawback, as the back (posterior) chamber of the eye is a frequent site of inflammation or otherwise the site of action where, ideally, ocular drug therapy should be targeted for many indications.

As an approach for circumventing the barriers encountered by local topical delivery, one local therapy route for the eye has involved direct intravitreal injection of a treatment drug through the sclera (i.e., the spherical, collagen-rich outer covering of the eye). However, the intravitreal injection delivery route tends to result in a short half-life and rapid clearance without sustained release capability being attained. Consequently, weekly to monthly injections are frequently required to maintain therapeutic ocular drug levels. This is not practical for many patients.

Given these drawbacks, the use of implant devices placed in or adjacent to the eye tissues to deliver therapeutic drugs thereto should offer a great many advantages and opportunities over the rival therapy routes. Despite the variety of ocular implant devices which have been described and used in the past, the full potential of the therapy route has not been realized. Among other things, prior ocular implant devices deliver the drug to the eye tissues via a single mode of administration for a given treatment, such as via slow constant rate infusion at low dosage. However, in many different clinical situations, such as with CNVM in AMD, this mode of drug administration might be a sub-optimal ocular therapy regimen.

Another problem exists with previous ocular implants, from a construction standpoint, insofar as preparation techniques thereof have relied on covering the drug pellet or core with a permeable polymer by multi-wet coating and drying approaches. Such wet coating approaches can raise product quality control issues such as an increased risk of delamination of the thinly applied coatings during subsequent dippings, as well as thickness variability of the polymer around the drug pellets obtained during hardening. Additionally, increased production costs and time from higher rejection rates and labor and an increased potential for device contamination from additional handling are known problems with present implant technology.

Accordingly, certain aspects of the present disclosure provide local treatment of a variety of eye diseases. Other aspects of the present disclosure also provide a method for the delivery of pharmaceuticals to the eye to effectively treat eye disease, while reducing or eliminating the systemic side effects of these drugs. Certain aspects of the present disclosure also provide shaped sustained-release ocular implants for administration of therapeutic agents to the eye for prolonged periods of time. Additionally, certain aspects of the present disclosure provide approaches to alter the areas of the eye that are affected by diffusion of drugs from sustained-release ocular implants. Certain aspects of the present disclosure also provide methods for making shaped ocular implants with reduced product variability.

In these and other ways described below, the implants of the present disclosure offer a myriad of advantages, improvements, benefits, and therapeutic opportunities. The implants are highly versatile and can be tailored to enhance the delivery regimen both in terms of administration mode(s) and type(s) of drugs delivered. The implants of this disclosure permit continuous release of therapeutic agents into the eye over a specified period of time, which can be weeks, months, or even years as desired. As another advantage, the implant systems of this disclosure require intervention only for initiation and termination of the therapy (i.e., removal of the implant). Patient compliance issues during a regimen are eliminated. The time-dependent delivery of one or more drugs to the eye by this disclosure makes it possible to maximize the pharmacological and physiological effects of the eye treatment. The implants have human and veterinary applicability.

### Multilayer Ocular Implant

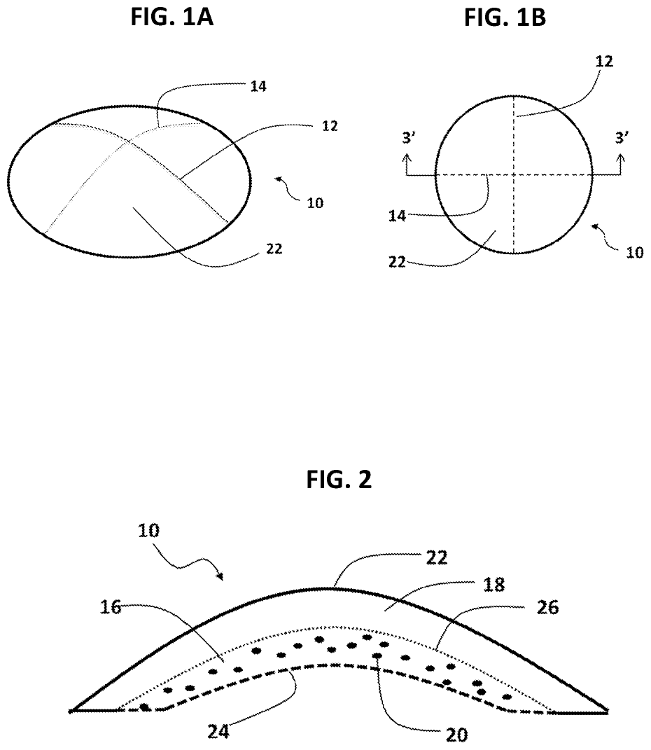

Certain embodiments of the ocular implant of the present disclosure are described herein, with reference to FIGS. 1 to 4. Skilled artisans will appreciate that elements in the figures are illustrated for simplicity and clarity and have not necessarily been drawn to scale. For example, the dimensions of some of the features shown in the figures may be enlarged relative to other elements to better illustrate and/or facilitate the discussion herein of the embodiments of the disclosure. Features in the various figures identified with the same reference numerals represent like features, unless indicated otherwise. Alternative features of alternative embodiments will also be discussed in context of the features of this example embodiment.

In certain embodiments of the present disclosure, the ocular implant is a multilayer ocular implant. In certain embodiments of the present disclosure, the ocular implant is a two-layer ocular implant. In certain embodiments, the ocular implant is a curved two-layer composite ocular implant. The curved shape of the implant 10 is indicated by dotted lines 12 and 14 in FIG. 1A and FIG. 1B. This shape may be formed by using a molding process, such as a molding process as taught in WO Patent Application 2014/179568, which is incorporated herein by reference in its entirety.

In certain embodiments, the ocular implant is formed by multiple (e.g. two) curved layers. In certain embodiments, the ocular implant is formed by a lower layer 16 and an upper layer 18 as can be seen in the cross-sectional view of FIG. 2 which is taken along line 3′-3′ of FIG. 1B. In certain embodiments, the lower layer 16 is formed from one or more biopolymers or composites thereof, which contains a therapeutic agent 20. The layers are demarcated by line 26 (FIG. 2). The lower layer 16 has a lower surface 24 which makes contact with the sclera E3 when the implant is in use.

In certain embodiments, the upper layer 18 is formed by one or more polymers, such as silicone polymers or other polymers. Examples of polymers suitable for forming the upper layer include, but are not limited to, polyvinyl acetate, cross-linked poly(vinyl alcohol), cross-linked poly(vinyl butyrate), ethylene ethylacrylate co-polymer, poly(ethyl hexylacrylate), poly(vinyl chloride), poly(vinyl acetals), plasiticized ethylene vinylacetate copolymer, poly(vinyl alcohol), poly(vinyl acetate), ethylene vinylchloride copolymer, poly(vinyl esters), polyvinylbutyrate, polyvinylformal, polyamides, poly(methyl methacrylate), poly(butyl methacrylate), plasticized poly(vinyl chloride), plasticized nylon, plasticized soft nylon, plasticized poly(ethylene terephthalate), natural rubber, polyisoprene, polyisobutylene, polybutadiene, polyethylene, polytetrafluoroethylene, poly(vinylidene chloride), polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer or vinylidene chloride-acrylonitride copolymer or any suitable equivalent of these polymers or combinations thereof. In certain alternative embodiments, the polymer is a silicone adhesive.

In certain embodiments, the lower layer 16 is formed by one or more polymers, such as medical grade biopolymers. In certain embodiments, the lower layer includes a polydimethylsiloxane (PDMS)-based compound. In certain embodiments, the lower layer includes a silicone adhesive. Silicone adhesives are generally biologically (physiologically) inert and is well tolerated by body tissues. Suitable silicones for use in implants of the present disclosure include MED-6810 silicone, MED1-4213, or MED2-4213 silicone. Other biocompatible silicone adhesives may be used and can be adapted for use in preparation of implants according to certain alternative embodiments of the present disclosure. The time and temperature needed to cure the silicone will depend on the silicone used and the drug release profile desired. These silicones, if left to cure at room temperature (e.g., 20-30° C.) will require about 24 hours or more to cure. The cure rate will increase with increasing cure temperatures. For instance, MED2-4213 silicone will cure in about 30 minutes at about 100° C. As will be discussed in more detail below, the more quickly the silicone is cured, the less opportunity for therapeutic agent to leach out of the layer. In some cases, a catalyst such as platinum may be used to induce curing.

In certain embodiments, the biocompatible polymer includes an ethylene-vinyl ester copolymer. In certain embodiments, the biocompatible polymer includes an ethylene-vinyl ester copolymer selected from: ethylene-vinyl acetate (EVA), ethylene-vinyl hexanoate (EVH), ethylene-vinyl propionate (EVP), ethylene-vinyl butyrate (EVB), ethylene vinyl pentantoate (EVP), ethylene-vinyl trimethyl acetate (EVTMA), ethylene-vinyl diethyl acetate (EVDEA), ethylene-vinyl 3-methylbutanoate (EVMB), ethylene-vinyl 3-3-dimethylbutanoate (EVDMB), ethylene-vinyl benzoate (EVBZ), or mixtures thereof. In certain embodiments, the biocompatible polymer includes an ethylene-vinyl acetate (EVA) copolymer.

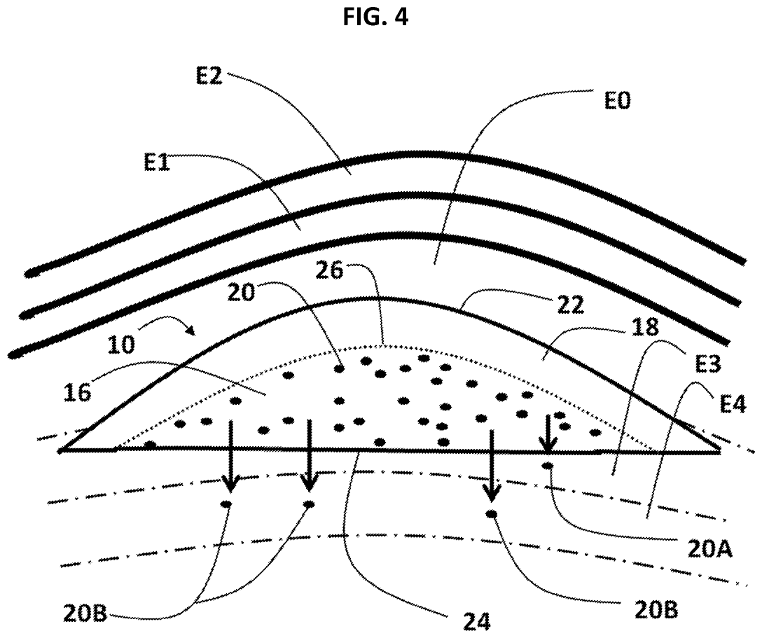

Dimensions of the ocular implant may vary. However, in this particular embodiment, the implant 10 has a diameter of 7 mm and a thickness of 2 mm. In this particular embodiment, each of the two layers 16 and 18 is 1 mm thick. In this particular embodiment, the upper surface 22 of the upper layer 18 has a radius of curvature of 5 mm for generally conforming to the radius of curvature of the surface of Tenon's capsule E1 of an average human eye (as indicated in FIG. 4). Likewise, the lower layer 16 is also curved with a similar radius of curvature configured to generally conform to the radius of curvature of the sclera E3 of an average human eye. These dimensions provide the implant 10 with characteristics appropriate for implantation with scleral contact in the sub-Tenon's space E0 of a human. It will be understood by the skilled person that these dimensions should be modified appropriately for an implant designed for use in an experimental animal such as a rat, mouse or rabbit for example. Armed with the knowledge of average dimensions of the eye and radii of curvature of Tenon's capsule and sclera of the chose experimental animal, the dimensions of an ocular implant according to may be selected by the skilled person and appropriate molding tools may be constructed without undue experimentation.

In certain embodiments, the ocular implant includes an upper layer 18 which is generally resistant to diffusion of the therapeutic agent 20 which is dispersed in the lower layer 16. In certain embodiments, the upper layer 18 is impermeable to the therapeutic agent 20. In other embodiments, the therapeutic agent 20 has a rate of diffusion within the upper layer 18 which is significantly less than the rate of diffusion of the therapeutic agent 20 out of the lower layer 16 and into the sclera. In this context, the term “significantly less” means 30%, 40%, 50%, 60%, 70%, 80%, 90% or 99% less than the rate of diffusion of the therapeutic agent 20 out of the lower layer 16 and into the sclera E3. The reduced diffusion characteristics of the therapeutic agent 20 in the upper layer 18 relative to the lower layer 16 provide the advantage of preventing loss of the therapeutic agent 20 to tissues where it is not needed. The reduced rate of diffusion of the therapeutic agent 20 through the upper layer 18 thereby encourages unidirectional diffusion of the therapeutic agent 20 from the lower layer 16 into the sclera E3 and choroid E4 for transfer to the macula E6 where its desired mechanism of action will be effected. A further advantage provided by the reduced diffusion characteristics of the therapeutic agent 20 in the upper layer 18 relative to the lower layer 16 is gained in preventing the therapeutic agent 20 from entering the lymphatic system via Tenon's capsule E1 and the conjunctiva E2 for transfer to other tissues where it may cause undesirable side-effects. Thus, in certain alternative embodiments of the present disclosure, the upper layer 18 or lower layer 16 further includes an agent that blocks lymphatic absorption.

In this particular embodiment, the thickness of the implant is 2 mm with the two layers 16 and 18 each being 1 mm in thick. The skilled person will appreciate that the thickness of each layer may be modified according to various embodiments of the disclosure, which may include variations with respect to the composition of silicone adhesive of the lower layer, the polymer of the upper layer, or the properties of drugs and/or formulations thereof used in the implant. The dimensional thickness may be modified appropriately by the skilled person without undue experimentation.

In certain embodiments, the therapeutic agent 20 in the lower layer 16 is an Nrf2 regulator such as sulforaphane, which is used in the treatment of macular degeneration. The drug is released over time as the drug particles 20 diffuse through the lower layer 16.

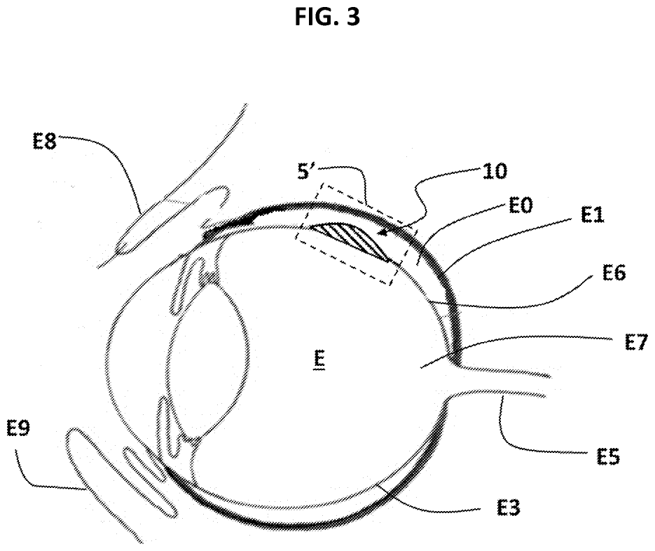

Positioning of the implant 10 with respect to the anatomical structures of an eye E is indicated in FIGS. 3 and 4. In FIG. 3, the features of the implant 10 are omitted for clarity. For convenient reference, the anatomical structures shown in FIGS. 3 and 4 include the sub-Tenon's space E0, Tenon's capsule E1 (also known as the bulbar sheath), the sclera E3, the choroid E4 (shown in FIG. 4 only), the optic nerve E5, the macula E6, the vitreous humor E7 and the upper and lower eyelids E8 and E9.

Referring now to FIG. 4 (which represents a magnification of the inset labeled 5′ in FIG. 3) there is provided additional detail regarding the placement of the implant 10. The implant 10 is located in the sub-Tenon's space E0 with its lower surface 24 resting upon the surface of the sclera E3. It is also seen that the upper surface 22 of the implant 10 has a curvature which generally conforms to the curvature of the surface of Tenon's capsule E1. This feature provides the advantage of minimizing discomfort to the eye as a result of contact of Tenon's capsule E1 with upper edges of the implant 10. The curved upper surface 22 is smooth and does not have sharp edges which would otherwise cause irritations and/or damage to the tissues of Tenon's capsule and possibly also the conjunctiva E2 in the event that a sharp edge of an alternative implant were to completely puncture Tenon's capsule E1 and penetrate the conjunctiva E2.

Particles of therapeutic agent 20 will be released downward to the sclera E3 as indicated by the arrows in FIG. 4, because they are concentrated in the lower layer 16 and because the upper layer 18 is generally resistant to diffusion of the therapeutic agent 20 as described above. In FIG. 4, it is shown that three drug particles 20B have diffused from the lower layer 16 through the sclera E3 to the choroid E4 and one drug particle 20A has diffused from the lower layer 16 to the sclera E3. These drug particles 20A and 20B are expected to be transferred by either diffusion or an active physiological mechanism, or a combination thereof, to the macula E6 where the desired pharmaceutical effect will be obtained. Notably, FIG. 4 does not include arrows indicating diffusion of the therapeutic agent 20 into the upper layer 18 and to upper tissues in Tenon's capsule E1 and the conjunctiva E2. This is due to resistance of the upper layer 18 to diffusion of the therapeutic agent 20.

In certain embodiments, the implant 10 is provided with a suture platform (not shown) which can be formed as part of the implant to facilitate attachment of the implant 10 to the sclera E3. An implant having a suture platform with a mesh contained therein to hold sutures in place is described in U.S. Pat. No. 7,658,364 (which is incorporated herein by reference in entirety). The implant described herein can be modified without undue experimentation to include such a suture platform by modification of the molding processes which will be described in detail hereinbelow. Alternatively, the implant of the disclosure may also be fixed to a suture stub as described also in U.S. Pat. No. 7,658,364.

In certain embodiments, the implant is circular or oval-shaped.

In certain embodiments, the outer layer is resistant to diffusion of the therapeutic agent from the silicone layer.

In certain embodiments, the outer layer is substantially impermeable to diffusion of the therapeutic agent from the silicone layer.

In certain embodiments, the outer layer and the inner layer are each about 1 mm thick.

In certain embodiments, the outer layer and/or the inner layer further include an agent that blocks lymphatic absorption of the therapeutic agent.

In certain embodiments, the inner layer further includes an ophthalmic permeation agent that increases ocular permeability of the therapeutic agent into the eye.

In certain embodiments, the ophthalmic permeation agent is methylsulfonylmethane.

In certain embodiments, the radius of curvature of the curved eye-contacting surface of the inner layer ranges from between about 5 mm to about 6 mm. In certain embodiments, the implant is circular with a diameter ranging between about 1 mm and 8 mm. In certain embodiments, the implant is circular with a diameter ranging between about 1 mm and 3 mm.

In certain embodiments, the implant includes a nutraceutical oil, such as omega-3 fish oil.

In certain embodiments, the silicone layer further includes an excipient that improves the release of drug. In certain embodiments, the excipient is selected from one or more of isopropyl myristate, levomenthol, propylene and tetraglycol.

In certain embodiments, the implant includes: a first hardened layer including a polymer, the first hardened layer including curvature at both surfaces; and a second hardened layer including a silicone adhesive and the therapeutic agent, the second hardened layer and including curvature at both surfaces.

In certain embodiments, the curvature of one surface of the first hardened layer and the curvature of one surface of the second layer are both formed using an impression body with a curved protrusion.

In certain embodiments, the first and second hardened layers are defined as follows: the curvature of a first surface of the first hardened layer is formed by dispensing the polymer into a mold body; the curvature of a second surface of the first hardened layer is formed by a first curved protrusion on a first impression body; the curvature of a first surface of the second hardened layer is formed by dispensing the silicone adhesive onto the curvature of the second surface of the first hardened layer; and the curvature of a second surface of the second hardened layer is formed by a second curved protrusion on a second impression body.

In certain embodiments, the first hardened layer is resistant to diffusion of the therapeutic agent from the second hardened layer.

In certain embodiments, the first hardened layer is substantially impermeable to diffusion of the therapeutic agent from the second hardened layer.

### III. Treatment and Uses

The implants and compositions of the present disclosure can be used to treat a number of eye diseases and indications including, for example, age-related macular degeneration, glaucoma, diabetic retinopathy, uveitis, retinopathy of prematurity in newborns, choroidal melanoma, chorodial metastasis, and retinal capillary hemangioma.

Age-related macular degeneration (AMD) is a common disease associated with aging that gradually impairs sharp, central vision. There are two common forms of AMD: dry AMD and wet AMD. About ninety percent of the cases of AMD are the dry form, caused by degeneration and thinning of the tissues of the macula; a region in the center of the retina that allows people to see straight ahead and to discern fine details. Although only about ten percent of people with AMD have the wet form, it poses a much greater threat to vision. With the wet form of the disease, rapidly growing abnormal blood vessels known as choroidal neovascular membranes (CNVM) develop beneath the macula. These vessels leak fluid and blood that destroy light sensing cells, thereby producing blinding scar tissue, with resultant severe loss of central vision. Wet AMD is the leading cause of legal blindness in the United States for people aged sixty-five or more with approximately 25,000 new cases diagnosed each year in the United States. Ideally, treatments of the indication would include inducing an inhibitory effect on the choroidal neovascularization (CNV) associated with AMD. The macula is located at the back of the eye and therefore treatment of CNVM by topical delivery of pharmacological agents to the tissues of the macula tissues is not possible. Intravitreal injections of anti-angiogenic agents, laser photocoagulation, photodynamic therapy, and surgical removal are currently used to treat CNVM. Unfortunately, the recurrence rate using such methods exceeds 50-90% in some cases. In most cases indefinite treatment is required.

Age related macular degeneration (AMD) is one of the major causes of vision loss in the elderly in most developed countries. Among many causes, oxidative stress in the retinal pigment epithelium (RPE) have been hypothesized to be a major driving force of AMD pathology. Oxidative stress could be treated by antioxidant administration into the RPE cells. However, to achieve high in-vivo efficacy of an antioxidant, it is imperative that the agent be able to penetrate the tissues and cells.

To administer the implant, the subconjunctival matrix implant can be is placed behind the surface epithelium within the sub-Tenon's space. This is done by a surgical procedure that can be performed in an out-patient setting. A lid speculum is placed and a conjunctival radial incision is made through the conjunctiva over the area where the implant is to be placed. Wescott scissors are used to dissect posterior to Tenon's fascia and the implant is inserted. The conjunctiva is reapproximated using a running 10-0 vicryl suture. The eye has many barriers that do not permit easy penetration of drugs. These include the surface epithelium on the front (cornea) of the eye and the blood/retinal barrier either within the retinal blood vessels or between the retinal pigment epithelium that both have tight junctions. These implants are generally about 1-2 mm in diameter for small rodent (i.e., mouse and rat) eyes, 3-4 mm in diameter for rabbit and human eyes and 6-8 mm in diameter for equine eyes.

The present disclosure provides a shaped ocular implant for delivery of drugs to the eye for treatment of diseases and disorders of the eye. In certain embodiments, the eye disorder is macular degeneration. In certain embodiments, the eye disorder is age-related macular degeneration (AMD).

In certain embodiments, an applicator device is used to inject the implant into the sub-Tenon's space. Such devices are known in the art and have been used for intraocular injections into the vitreous humor of the eye, particularly in intraocular lens implantation after cataract surgery. In certain embodiments, the device is provided with a retractor that engages the conjunctiva and the surface of Tenon's capsule to produce an opening into the sub-Tenon's space. The device is also provided with a means for pushing the implant into the sub-Tenon's space such that withdrawal of the device allows the surrounding tissues to collapse back into place while holding the implant at the desired location.

Additionally, when the implant is placed near the limbus (i.e., the area where the conjunctiva attaches anteriorly on the eye) to encourage the drug diffusion to enter the cornea, it may be possible to fixate the matrix implant with one or two absorbable sutures (e.g., 10-0 absorbable vicryl sutures). This is done by making holes with a 30 gauge needle in the peripheral portion of the implant, approximately 250-500 μm away from the peripheral edge of the implant. The holes are made 180 degrees from each other. This is done because subconjunctival matrix implants of this disclosure, when placed near the cornea, are at higher risk to extrude because of the action of the upper eye lid when blinking. When subconjunctival matrix implants of this disclosure are placed about 4 mm or more away from the limbus, the sutures are optional.

This matrix implant can deliver therapeutic levels of different pharmaceuticals agents to the eye to treat a variety of diseases. Using a rabbit model, drug released from the implant placed in the eye produces negligible levels of the drug in the blood. This significantly reduces the chances of systemic drug side-effects. This implant design of this disclosure is prepared by unique methodologies and selections of materials leading to and imparting the unique pharmacological performance properties present in the finished devices.

In certain embodiments, the present implants provide a sustained or controlled delivery of therapeutic agents at a maintained level despite the rapid elimination of the lipophilic agents from the eye. For example, the present implants are capable of delivering therapeutic amounts of a lipophilic agent for a period of at least about 30 days to about a year despite the short intraocular half-lives associated with lipophilic agents. The controlled delivery of lipophilic agents from the present implants permits the lipophilic agents to be administered into an eye with reduced toxicity or deterioration of the blood-aqueous and blood-retinal barriers, which may be associated with intraocular injection of liquid formulations containing lipophilic agents.

The implants may be placed in an ocular region to treat a variety of ocular conditions, such as treating, preventing, or reducing at least one symptom associated with non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, retinitis pigmentosa, ocular tumors, ocular neoplasms, and the like.

Kits in accordance with the present disclosure may include one or more of the present implants, and instructions for using the implants. For example, the instructions may explain how to administer the implants to a patient, and types of conditions that may be treated with the implants.

### IV. Definitions

At various places in the present disclosure, substituents or properties of compounds of the present disclosure are disclosed in groups or in ranges. It is specifically intended that the present disclosure include each and every individual or sub-combination of the members of such groups and ranges.

Unless stated otherwise, the following terms and phrases have the meanings described below. The definitions are not meant to be limiting in nature and serve to provide a clearer understanding of certain aspects of the present disclosure.

About: As used herein, the term “about” means+/−10% of the recited value.

Activity: As used herein, the term “activity” refers to the condition in which things are happening or being done. Compositions of the present disclosure may have activity and this activity may involve one or more biological events.

Associated: As used herein, the terms “associated” or “associated with” mean mixed with, dispersed within, coupled to, covering, or surrounding.

Administering: As used herein, the term “administering” refers to providing a pharmaceutical agent or composition to a subject.

Administered in combination: As used herein, the term “administered in combination” or “combined administration” means that two or more agents are administered to a subject at the same time or within an interval such that there may be an overlap of an effect of each agent on the patient. In certain embodiments, they are administered within about 60, 30, 15, 10, 5, or 1 minute of one another. In certain embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals include, but are not limited to, mammals, birds, reptiles, amphibians, fish, and worms. In certain embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biocompatible: As used herein, the term “biocompatible” or “bioerodible” mean compatible with living cells, tissues, organs or systems posing little to no risk of injury, toxicity or rejection by the immune system.

Biodegradable: As used herein, the terms “biodegradable” means capable of being broken down into innocuous products by the action of living things. The term “biodegradable polymer” refers to a polymer or polymers which degrade in vivo, and wherein degradation of the polymer or polymers over time occurs concurrent with or subsequent to release of the therapeutic agent. Specifically, hydrogels such as methylcellulose which act to release drug through polymer swelling are specifically excluded from the term “biodegradable polymer”. A biodegradable polymer may be a homopolymer, a copolymer, or a polymer including more than two different polymeric units.

Controlled release: As used herein, the term “controlled release” refers to a pharmaceutical composition or compound release profile that conforms to a particular pattern of release to affect a therapeutic outcome.

Depression: As used herein, the term “depression” refers to a region of a surface which is lower with respect to the majority of the surface. More specifically, the present specification describes a depression in a mold body which represents a region with a lower surface than the remainder of the contact surface of the mold body.

Encapsulate: As used herein, the term “encapsulate” means to enclose, surround or encase.

Effective amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Formulation: As used herein, a “formulation” includes at least one therapeutic agent and a delivery agent or excipient.

Impression body: As used herein, the term “impression body” refers to a body used to alter a surface of another body by pressure. The impression body may have one or more features that produce an impression having a specific shape such as a curvature for example.

Nutraceutical: As used herein, the term “nutraceutical” refers to an isolated nutrient that may have therapeutic benefit against a disease or disorder. A non-limiting example of a nutraceutical oil is an omega-3 fish oil.

Ocular condition: As used herein, an “ocular condition” is a disease, ailment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball.

An “anterior ocular condition” is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the retina but in front of the posterior wall of the lens capsule), the lens or the lens capsule and blood vessels and nerve which vascularize or innervate an anterior ocular region or site. Thus, an anterior ocular condition can include a disease, ailment or condition, such as for example, aphakia; pseudophakia; astigmatism; blepharospasm; cataract; conjunctival diseases; conjunctivitis; comeal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A “posterior ocular condition” is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site. Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

Ocular implant: As used herein, the terms “ocular implant” or “intraocular implant” refer to a device or element that is structured, sized, or otherwise configured to be placed in an eye. Ocular implants are generally biocompatible with physiological conditions of an eye and do not cause adverse side effects. Ocular implants may be placed in an eye without disrupting vision of the eye.

Ocular region: As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina.

Ophthalmic permeation agent: As used herein the terms “ophthalmic permeation agent” or “transport facilitator” refer to a compound that increases the permeability of a therapeutic agent into the tissues of the eye. Methylsulfonylmethane is a non-limiting example of an ophthalmic permeation agent.

Patient: As used herein, “patient” refers to a subject who may seek or need treatment, requires treatment, is receiving treatment, will receive treatment, or a subject who is under care by a trained professional for a particular disease or condition.

Permeation agent: As used herein, the term “permeation agent” refers to a molecule that increases the permeability of a therapeutic agent. An ophthalmic permeation agent increases the permeability of a therapeutic agent with respect to tissues of the eye.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile can be used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety insofar as they do no conflict with the present disclosure.

Pharmaceutically acceptable solvate: The term “pharmaceutically acceptable solvate,” as used herein, means a compound of the present disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Radius of curvature: As used herein the term “radius of curvature” refers to the radius of a circle that best fits the curved surface at a given point.

Stable: As used herein “stable” refers to a compound that is sufficiently robust to survive isolation to a useful degree of purity from a reaction mixture, and in certain embodiments, capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In certain embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Therapeutic agent: The term “therapeutic agent” refers to any agent that, when administered to a subject, has a therapeutic, diagnostic, and/or prophylactic effect and/or elicits a desired biological and/or pharmacological effect.

Therapeutic composition: As used herein, the terms “therapeutic composition” or “therapeutic component” refer to a portion of formulation or an implant which includes one or more therapeutic agents or substances used to treat a medical condition, such as a medical condition of the eye.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In certain embodiments, a therapeutically effective amount is provided in a single dose. In certain embodiments, a therapeutically effective amount is administered in a dosage regimen including a plurality of doses. Those skilled in the art will appreciate that in certain embodiments, a unit dosage form may be considered to include a therapeutically effective amount of a particular agent or entity if it includes an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24-hour period. It may be administered as a single unit dose.

Treating: As used herein, the terms “treat”, “treating” or “treatment” refer to partially or completely alleviating, ameliorating, improving, reducing, resolving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

### V. Equivalents and Scope

Those skilled in the art will recognize or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### EXAMPLES

### Example 1. Evaluation of NAC alkyl-ester analogues as prodrugs

Five (5) lipophilic cysteine prodrugs were evaluated for protecting human retinal pigment epithelial cells from oxidative stress induced by hydroquinone (HQ). The lipophilic cysteine prodrugs were: N-acetylcysteine (NAC), N-acetylcysteine methyl ester (NACME), N-acetylcysteine ethyl ester (NACEE), N-acetylcysteine propyl ester (NACPE), and N-acetylcysteine butyl ester (NACBE). To mimic in vitro AMD conditions, hydroquinone was used as the oxidative insult.

Cytosolic and mitochondrial protection against oxidative stress were tested using cytosolic and mitochondrial specific assays. The results provide evidence that these lipophilic cysteine prodrugs provide increased protection against oxidative stress in human RPE cells compared with NAC.

Viability of ARPE-19 cells were measured by XTT assay after pretreating the cells with the prodrugs followed by treating with HQ. Conversion of NAC prodrugs to NAC, cysteine and then to glutathione (GSH) was monitored through high performance liquid chromatography (HPLC) and GSH assay. Due to the strong correlation between age related macular degeneration and damage to mitochondria, the efficacy of the prodrugs towards protecting mitochondria from oxidative damage was evaluated using mitochondrial specific assays.

### Synthesis of N-Acetylcysteine (NAC) Alkyl-Ester Analogues

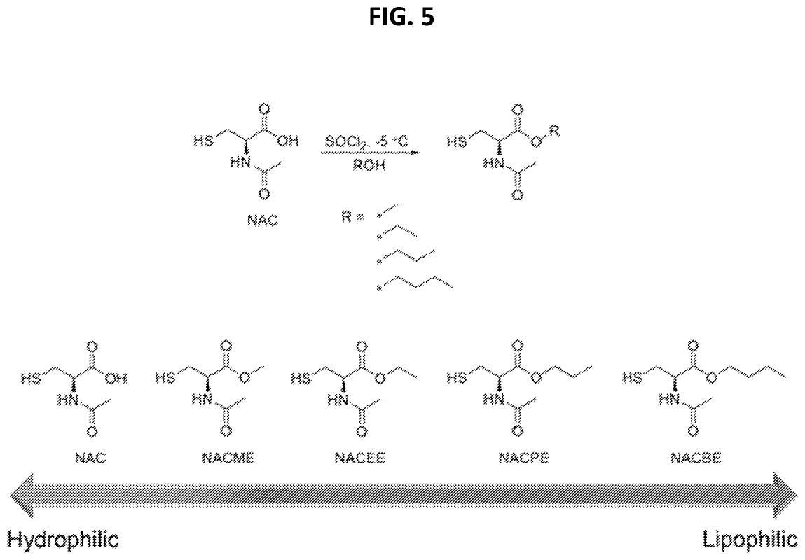

The NAC alkyl-ester analogues were synthesized by conversion of the carboxylic acid group in NAC to acyl chloride and then subsequent esterification with an appropriate alcohol (FIG. 5). NAC (1.00 g, 6.13 mmol) was dissolved in the appropriate alcohol (methanol, ethanol, propanol or butanol, 12.0 mL) under an argon atmosphere. For propanol and butanol, NAC was allowed to dissolve overnight. However, only a suspension was obtained. The solution was cooled to −5° C., and thionyl chloride (0.53 mL, 7.31 mmol) was added drop wise into the stirring solution. The reaction was stirred for 15 minutes at −5° C. and at room temperature for 2 hours. Solvent was removed under reduced pressure and the resulting slurry was extracted with ethyl acetate and washed with deionized (DI) water.

All compounds were purified with column chromatography using silica gel. NACME was obtained as a white solid upon removing the solvent under reduced pressure. NACEE, NACPE, and NACBE were subjected to column chromatography using silica gel (Eluents: NACEE: 100% ethyl acetate, NACPE: hexanes: ethyl acetate 1:2 v/v, NACBE: 100% hexanes to remove excess butanol, followed by hexanes: ethyl acetate 3:2 v/v). All compounds were obtained as colorless oils which solidified upon storage at −20° C. to afford off-white solids. Pure compounds were obtained in moderate yields and were characterized with ¹H and ¹³C NMR spectroscopy. The synthesized compounds have increasing lipophilicity from NACME to NACBE (FIG. 5) with the increase in the number of carbon atoms.

### ARPE-19 Cell Culture

ARPE-19 cells were grown in DMEM:F-12 supplemented with 10% fetal bovine serum (FBS). For all experiments these cells were split and grown in 6-well plates using MEM-Nic supplemented with 1% FBS according to a previously published procedure.³³ Cells used for all the experiments were between passages 25-30. For all experiments, the 96 well plates and 8-well slides were coated with 0.039 mg/mL collagen I at 6 μg/cm². The cells were seeded at a cell density of 70,000 cells/well and 150,000 cells/well using MEM-Nic media for 96 well plates and 8 well slides, respectively. Once the cells are confluent, media was replaced with MEM a, GlutaMAX™, supplemented with 1% FBS for 24 hours before carrying out assay protocols. Exposure to NAC alkyl-ester prodrugs were carried out in MEM a, GlutaMAX™, supplemented with 1% FBS and treatment with HQ was carried out in serum free DMEM:F-12.

### XTT Cell Viability Assay

ARPE-19 Cell viability assays were carried out using XTT/PMS reagent mixtures according to standard procedures known in the art (see Celis, J. E.; Carter, N., Cell Biology: A Laboratory Handbook. Elsevier Science: 2005). Corresponding absorbance readings were obtained using a plate reader at 450 and 660 nm.

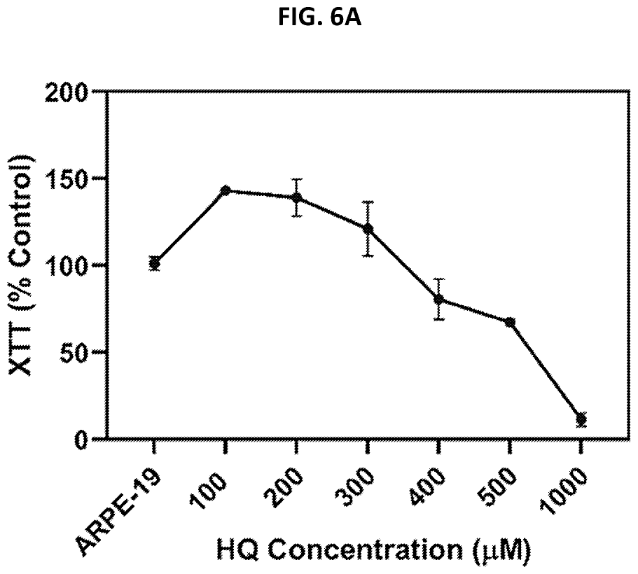

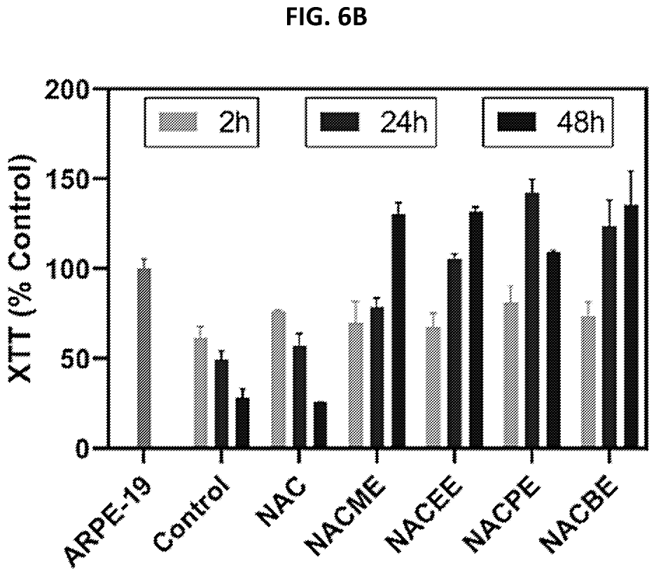

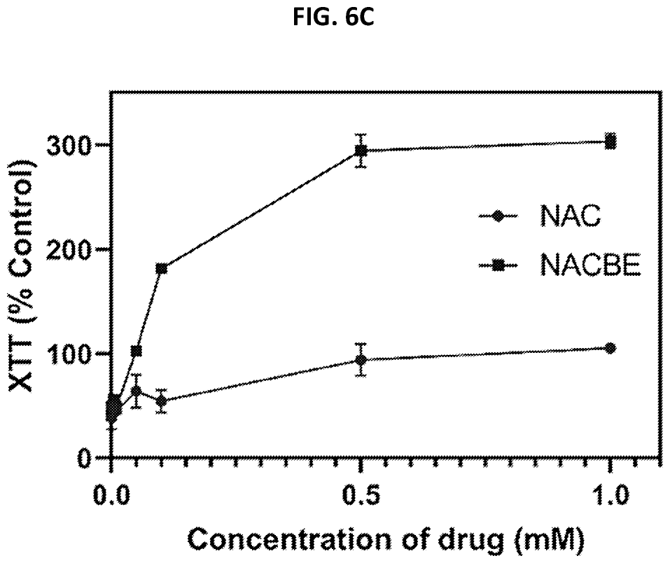

A dose dependent study was first carried out for ARPE-19 cells using HQ concentrations varying from 100-1000 μM. Cells were then incubated at 37° C., 5% CO₂ for 16 hours. Results are shown in FIG. 6A. HQ doses of less than 400 μM were non-lethal. Using 500 μM HQ for 16 hours gave a cell viability of ˜60%.

To evaluate the ability of the NAC alkyl-ester analogues to provide cellular protection against oxidative stress, cells were treated with 0.05 mM of NAC, NACME, NACEE, NACPE and NACBE for 2, 24 and 48 hours. Treated cells were then exposed to 500 μM HQ for 16 hours. HQ solutions were removed and replaced with DMEM:F-12 supplemented with 1% FBS, and followed by the addition of XTT (2,3-bis-(2-methoxy-4-nitro-5-sulfophenyl)-2H-tetrazolium-5-carboxanilide) and PMS (phenazine methyl sulfate). Assay results are shown in FIG. 6B. At a pretreatment time of 2 hours, no change in cell viability was observed for any drug upon exposure to HQ. However, with increasing pretreatment time to 24 and 48 hours, a significant increase in cell viability was observed for the NAC alkyl-ester analogues compared to NAC and the control.

To compare the effectiveness of NACBE and NAC in protecting against oxidative damage, another dose dependent study was carried out by varying the pretreatment concentration from 0.001 mM to 1.0 mM. As seen from FIG. 6C, when the concentration of NACBE reached 0.05 mM, a 100% cell viability was obtained. Whereas for NAC, to reach the same cell viability, 0.5 mM concentration was needed (10× times that of NACBE).

The XTT cell viability assay study showed that a low pretreatment time was ineffective for all NAC alkyl-ester analogues (with only NACBE showing marginal effect), as these compounds need more incubation time in order to undergo hydrolysis and eventually to synthesize GSH. Increasing incubation time from 2 hours to 24 and 48 hours showed a significant improvement in cell viability. Overall, NACBE showed comparatively a higher cell viability thereby providing the most protection against the introduced insult. The dose responsive behavior of NAC and NACBE also showed the effectiveness of NACBE towards protecting cells from oxidative damage compared to NAC.

### ZO-1 Staining

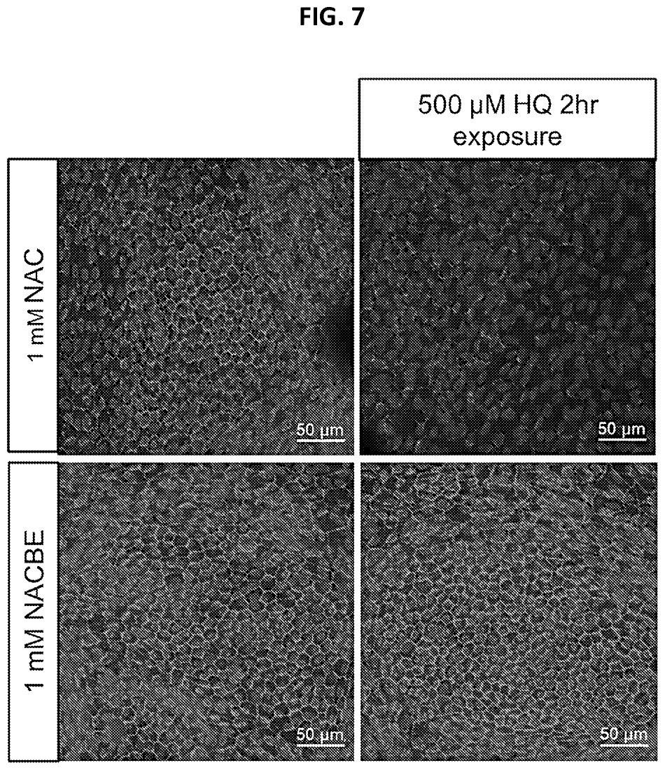

ARPE-19 cells were grown on an 8-well slide until confluent. The cells were exposed to 1 mM NAC and NACBE for 24 hours, followed by treatment with 500 μM of HQ for 2 hours. The cells were washed with 3 cycles of PBS, and then fixed with 4% Paraformaldehyde at 4° C. for 30 mins. After fixation the cells were blocked in PBST (0.2% Triton X-100)+1% BSA for 60 mins. Primary antibody (rabbit anti-Zo-1, Invitrogen) was diluted 1/100 in PBST+1% BSA and added overnight at 40 C. Cells were washed ×3 with PBS and secondary antibody (Donkey anti-Rabbit AF-555, Abcam) was added for 4 hours at RT. Cells were washed ×3 with PBS and mounted with Prolong Diamond Mountant with DAPI (Invitrogen). Results from ZO-1 staining are shown in FIG. 7.

Cells treated with NAC and NACBE exhibit proper cell-cell junctions (FIG. 7, left column). After exposure to HQ (FIG. 7, right column), ZO-1 staining present in cells treated with NAC diminished or was completely absent. For the cells pretreated with NACBE, the cellular junctions were intact even after the exposure to HQ.

The ZO-1 Staining study demonstrated the protection given by NACBE compared to NAC. Exposing ARPE-19 cells to HQ disrupted the cellular junctions due to the production of ROS. Introduction of antioxidants such as NACBE provided protection from the excess ROS produced by the insult. As a result, the cellular junctions were left intact, as visualized by the ZO-1 staining.

### HPLC Analysis

Without being bound by theory, NAC alkyl-ester analogue pro-drugs are predicated to undergo hydrolysis through cellular processing and are thus expected to increase the intracellular levels of NAC.

ARPE-19 cells were seeded onto a 60 cm² dish and was allowed to grow to confluency. The cells were treated with 1 mM NAC and NACBE for 1 hour in HBSS. The drug solution was aspirated and washed twice with HBSS. The cells were scraped with the aid of methanol (˜1 mL) and collected into 2 mL centrifuge tubes. The cell suspension in methanol was sonicated (for cell lysis) in a water bath for 30 minutes and was centrifuged at 14,000 RPM for 15 minutes. The supernatant was transferred to a HPLC vial and methanol was evaporated under a stream of nitrogen.

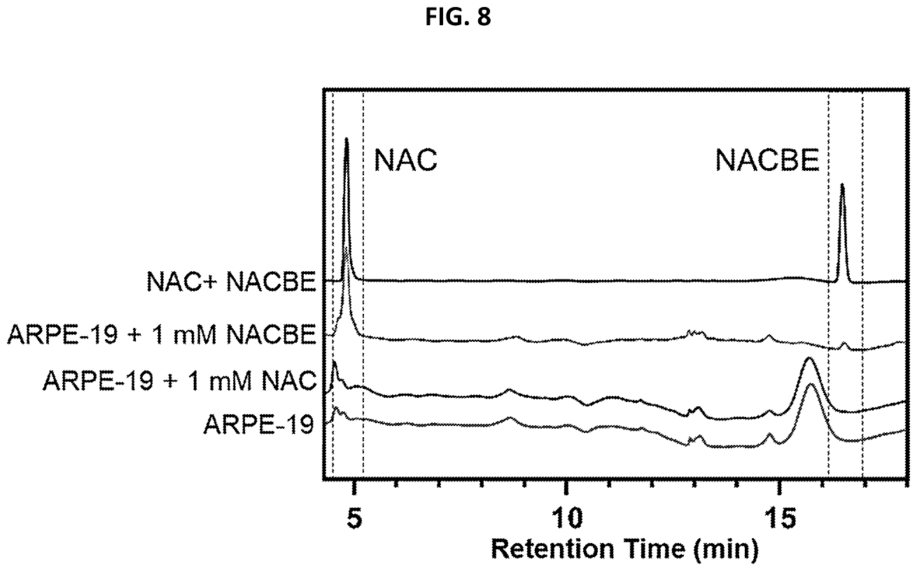

The sample was resuspended in 50 μL of methanol before injecting into the HPLC system. Samples and standard (20 μL) were injected with an autosampler. Separation was conducted by 0.8 mL/min gradient elution with a water/0.10% formic acid and acetonitrile mobile phase on a 250×4.6-mm (5-mm) C18 column (Restek, Pinnacle II) maintained at 25° C. The samples were monitored at 205 nm with a UV detector and analyzed with Agilent Chemstation software. Results are shown in FIG. 8.

For ARPE-19 cells treated with 1 mM NAC, no NAC was detected and the HPLC chromatogram was identical to ARPE-19 cells only. In contrast, when ARPE-19 cells were treated with 1 mM NACBE for 1 hour, HPLC analysis demonstrated both NACBE (prodrug) and NAC (metabolite) within the cells.

The HPLC analysis study confirmed the conversion of NACBE to NAC as well as the cellular uptake. As shown in FIG. 8, the NACBE is taken up by the cells more effectively than NAC and is shown to undergo intracellular conversion to NAC.

### GSH Assay

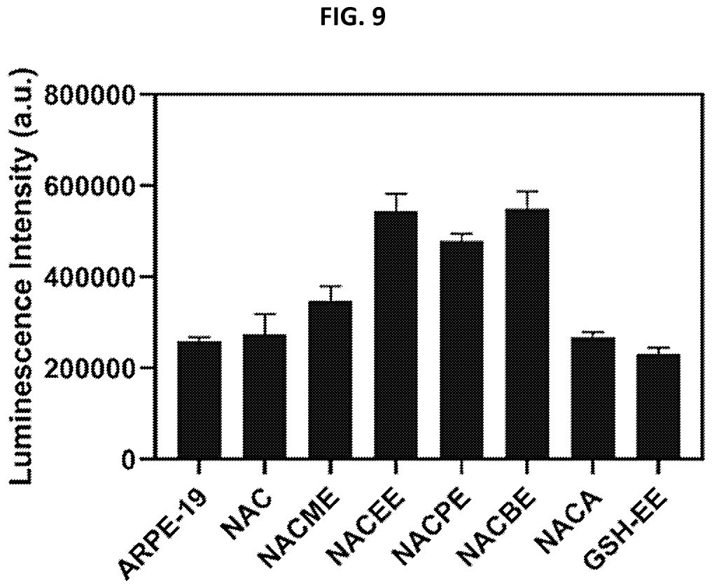

The production of cellular GSH levels upon exposure to drugs were measured using the GSH assay kit. NACA and GSH-EE were used as controls in this assay. ARPE-19 cells were grown in white 96 well plates. The cells were exposed to 1 mM solutions of NAC, NACME, NACEE, NACPE, NACBE, NACA and glutathione ethyl ester (GSH-EE) for 24 hours. The solutions were removed and washed with PBS once. Afterwards, GSH assay was carried out according to manufacturer recommended protocol (Promega GSH/GSSG-Glo™) and luminescence readings were obtained with a luminometer, which a higher luminescence intensity indicates a higher GSH concentration. Results are shown in FIG. 9.

Results show that NACEE, NACPE and NACBE produced the highest amounts of GSH compared to untreated ARPE-19 cells. The parent compound, NAC, and the positive controls, NACA and GSH-EE, did not produce any significant GSH compared to the untreated cells.

The GSH assay study showed the ability of the NAC alkyl-ester analogue pro-drugs to facilitate the generation of higher levels of GSH in target cells.

Confocal Microscopy with Dansyl Tagged N-Acetylcysteine Esters

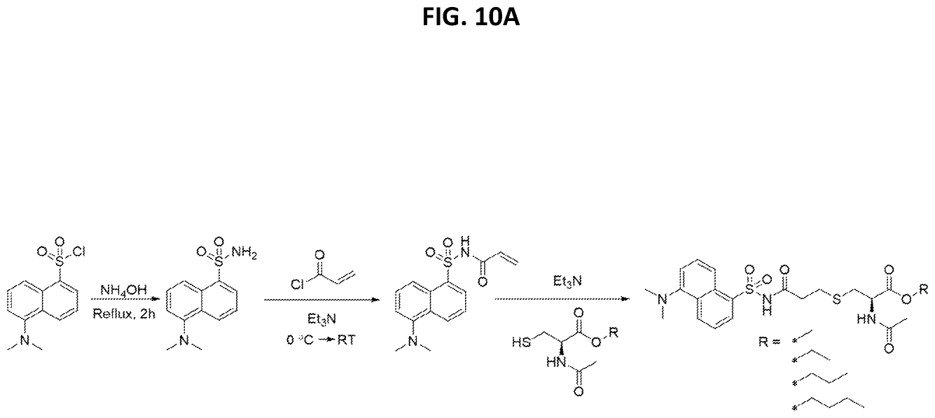

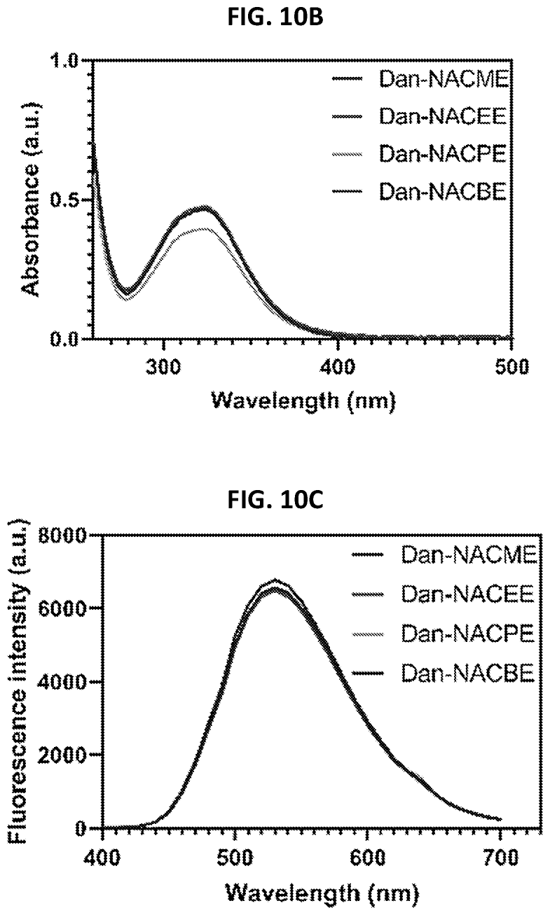

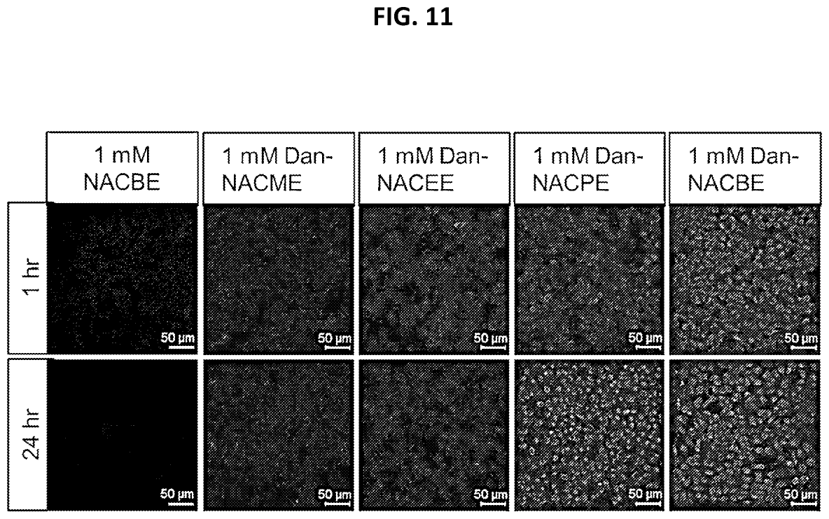

Dansyl-tagged NAC alkyl-ester analogues were synthesized, as shown in FIG. 10A. Dansyl chloride was reacted with ammonium hydroxide to yield dansyl amide. Then the dansyl probe: N-((5-(dimethylamino)-1-naphthalen-1-yl)sulfonyl)acrylamide was synthesized by reacting dansyl amide and acryloyl chloride. Both dansyl amide and N-((5-(dimethylamino)-1-naphthalen-1-yl)sulfonyl)acrylamide were obtained in good yields and were characterized with ¹H NMR spectroscopy. NACME, NACEE, NACPE and NACBE were then reacted with N-((5-(dimethylamino)-1-naphthalen-1-yl)sulfonyl)acrylamide in the presence of triethylamine to give Dan-NACME, Dan-NACEE, Dan-NACPE and Dan-NACBE respectively (FIG. 10A). All dansyl tagged compounds were characterized using ¹H and ¹³C NMR spectroscopy. All compounds possessed similar absorption and fluorescence profiles with λex ˜320 nm and λem ˜520 nm (FIG. 10B and FIG. 10C).

ARPE-19 cells were grown in 8 well slides before exposing to 1 mM solutions of Dan-NACME, Dan-NACEE, Dan-NACPE and Dan-NACBE for 1 and 24 hours. NACBE was used as the control. The cells were washed twice with PBS followed by mounting using PBS. Confocal images were obtained in the DAPI channel at 20× magnification.

At 1-hour incubation with the dansyl tagged NAC alkyl-ester analogues, the fluorescence intensity was shown to increase with increasing compound lipophilicity. The intensities further improved upon extending the incubation time to 24 hours, and Dan-NACBE had the highest fluorescence intensity.

### JC-1 Assay

JC-1 assays measuring the change in mitochondrial membrane potential were carried out to evaluate the protection of the NAC alkyl-ester analogues towards mitochondrial damage.

Typically, JC-1 dye has an inherent green fluorescence at 530 nm. Upon reaching the cell, due to the structural properties of the dye, it will accumulate in the mitochondria making aggregates known as J-aggregates. These J-aggregates consists of a red shifted fluorescence (590 nm). Damaged or unhealthy mitochondria, due to their depolarized membrane potential (compared to healthy ones), will have lesser amounts of aggregates and thus will have low intensity of red emission. Cells with healthy mitochondria will have a more prominent red fluorescence than that in cells with damaged/unhealthy mitochondria due to the ability in forming J-aggregates.

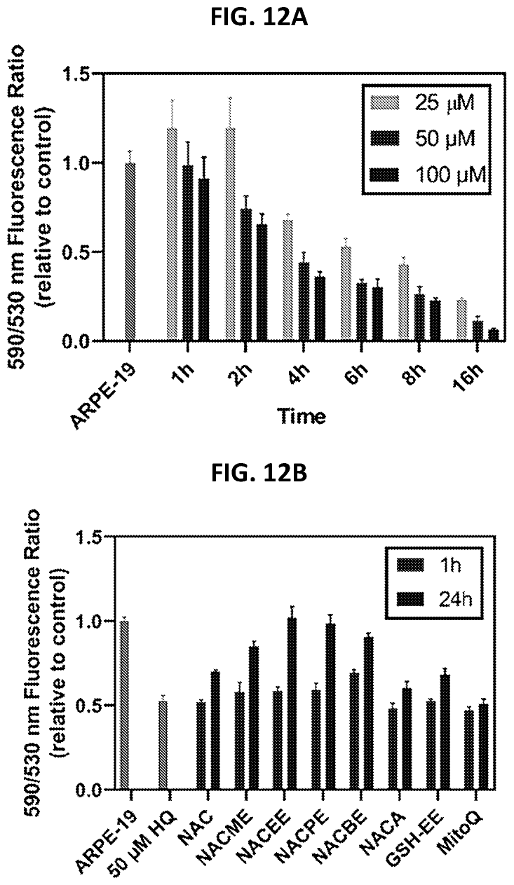

ARPE-19 cells were grown in black, clear bottom 96 well plates. A dose dependent study was carried out using 25, 50 and 100 μM HQ at 1, 2, 4, 6, 8 and 16-hour time points to determine the dose and time of the insult (FIG. 12A). With increasing time and dose of HQ, a drop in 590 nm/530 nm fluorescence is seen due to the depolarization of the mitochondria. For the assay 50 μM HQ for 4 hours was used as the dose and time for the insult, as it this combination was shown to exhibit moderate depolarization compared to the control.

Next, to assess the effect of NAC alkyl-ester analogues on oxidative damage in mitochondria (FIG. 12B), ARPE-19 cells were pretreated with 1 mM NAC, NAC alkyl-ester analogues, NACA, GSH-EE and 1 μM MitoQ for 1 and 24 hours. The cells were then exposed to 50 μM HQ for 4 hours. The HQ solutions were removed and washed once with PBS before the addition of JC-1 reagent. 10 μM solution of JC-1 reagent in serum free DMEM:F-12 was prepared by diluting 1 mM JC-1 solution in DMSO. The 10 μM solution was centrifuged at 7,200 g for 5 minutes before the addition to the cells followed by incubating at 37° C., 5% CO₂ for 30 minutes. The JC-1 solution was removed and washed once with PBS and fluorescence measurements were obtained in PBS at 485 nm excitation and emission at 535 nm and 590 nm. For consistency, NACA and GSH-EE were used as the positive controls. Since these molecules are not targeted towards mitochondria, MitoQ (1 μM), a well-known mitochondrial targeted antioxidant, was selected as an additional positive control.

Results at an incubation time of 1 hour showed that only NACBE demonstrated improved protection towards mitochondrial damage (relative to control). Upon extending the incubation time to 24 hours, all NAC alkyl-ester analogues were able to protect mitochondrial depolarization caused by HQ, while the parent compound and all the positive controls failed to show any additional protection.

### JC-1 Staining

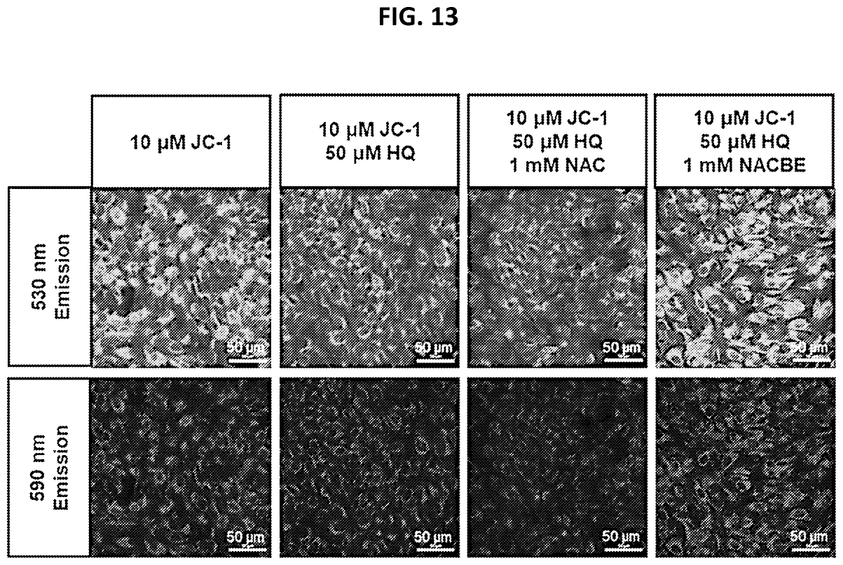

Results from the JC-1 assay were confirmed by JC-1 staining (FIG. 13). Confluent ARPE-19 cells were pretreated with the drugs for 24 hours before exposing to 50 μM HQ for 4 hours. Following HQ treatment, a 10 μM solution of JC-1 was added to the cells for 30 minutes, the cells were washed with PBS×3 and then mounted in Antifade Mountant (Invitrogen). The cells were imaged on the confocal (Zeiss LSM 800) by excitation with the 488 nm laser and emission imaged at 530 nm (green channel) and 590 nm (red channel).

Results showed that treatment of ARPE-19 cells with 50 μM HQ (FIG. 13, second column) provided a decreased emission intensity at 590 nm compared to that of untreated ARPE-19 cells (FIG. 13, first column). Pretreatment with 1 mM NAC did not help to retain mitochondrial depolarization with the introduction of the insult, shown by a similar reduction in the fluorescence intensity (FIG. 13, third column). In contrast, pretreatment with 1 mM NACBE preserved the mitochondrial membrane potential, showing a similar fluorescence intensity as the untreated ARPE-19 cells (FIG. 13, fourth column).

### Mitochondrial GSH Assay

A GSH assay was carried out for isolated mitochondria to study the mechanism of action of the NAC alkyl-ester analogues in protecting mitochondria. ARPE-19 cells were grown in 6 well plates until 100% confluent in MEM-NIC media. The cells were exposed to 3 mL of 1 mM solutions of NAC and NACBE for 24 hours. The solutions were removed and washed with 3 mL of HBSS before adding 1 mL of 0.25% Trypsin-EDTA and incubating for 10 minutes. 2 mL of DMEM:F-12 supplemented with 10% FBS was added to each well, harvested and centrifuged at 300 rcf for 5 minutes. The supernatant was removed and cell pellet was resuspended in 2 mL of isolation buffer (0.25 M sucrose and 10 mM HEPES).

Cells were disrupted using a probe sonicator (Misonix 5-3000) for 10 seconds in ice. Subsequently, intact cells and debris were removed by centrifuging at 1000 g for 10 mins. Supernatant was collected, and centrifuged at 20,000 g for 25 minutes. Pellet containing mitochondria were saved and washed using 0.5 mL of isolation buffer. After centrifuging at 20,000 g for 25 minutes the mitochondrial pellet was resuspended in 50 μL of HBSS. 25 μL was used to determine total GSH and GSSG and 25 μL was used to determine GSSG levels. GSH assay was then carried out according to manufacturer recommended protocol (Promega GSH/GSSG-Glo™) and luminescence readings were obtained.

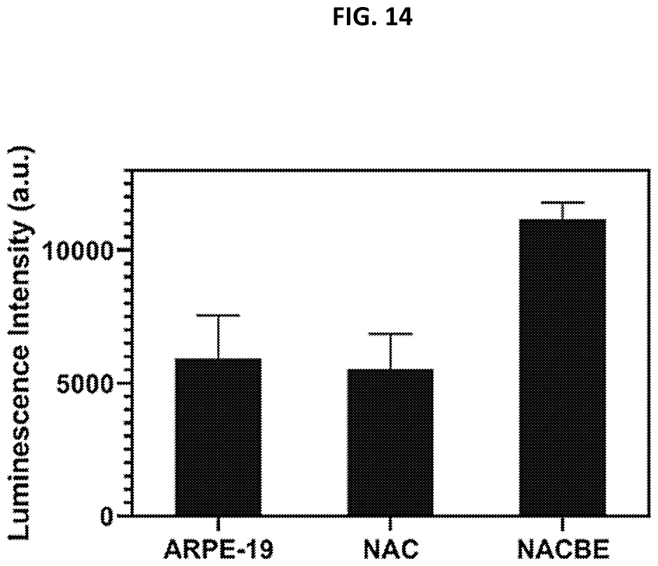

Results are shown in FIG. 14. Results show that mitochondria isolated from cells treated with NACBE showed an increase in the luminescence intensity compared to NAC and ARPE-19 cells. A higher luminescence intensity indicates a higher GSH level.

### CellTiter-Glo Assay

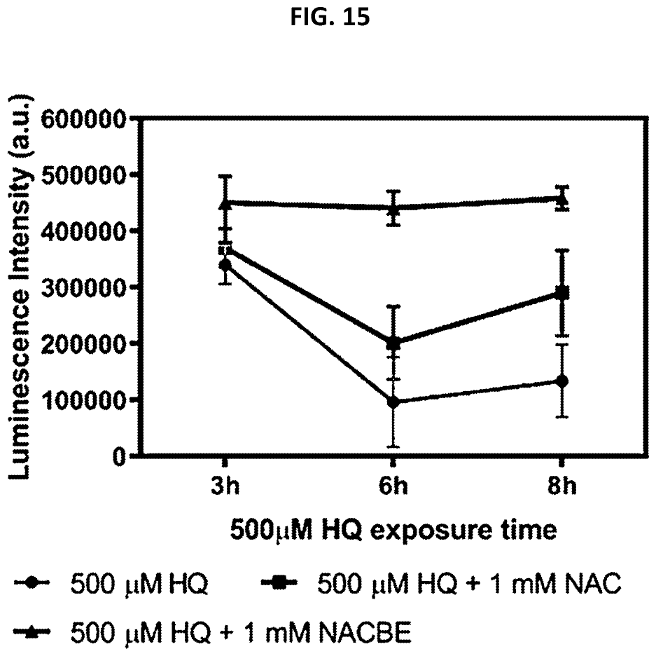

ARPE-19 cells were grown in white 96 well plates. The cells were first exposed to 500 μM of HQ for 3, 6 and 8 hours and the amount of ATP produced was measured using the CellTiter-Glo assay kit to obtain a time dependent response (FIG. 15). With increasing incubation time, the level of ATP decreased, which is indicative of the reduced luminescence intensity. Due to the mitochondrial damage caused by HQ, the production of ATP was decreased. Results showed that pretreatment with 1 mM NAC provided some protection to the introduced insult. However, with the use of 1 mM NACBE, the ATP production remained unaltered.

### DNA Fragmentation Assay

Mitochondrial DNA damage has been linked to pathogenic diseases, including AMD. To determine if pretreatment of RPE cells with NAC alkyl-ester analogues could protect mitochondrial DNA against oxidative damage, a long-extension PCR based assay was used to measure amplification of a large stretch of mitochondrial DNA. The mitochondrial DNA damage assay was performed according to protocols known to those in the art (see Santos, J. H.; Mandavilli, B. S.; Van Houten, B., Measuring Oxidative mtDNA Damage and Repair Using Quantitative PCR. In Mitochondrial DNA: Methods and Protocols, Copeland, W. C., Ed. Humana Press: Totowa, N.J., 2002; pp 159-176). Briefly, ARPE-19 cells were grown to confluency in 6-well plates. The cells were treated with NAC alkyl-ester analogues for 24 hours, washed and then treated with 500 uM HQ for an additional 24 hours. DNA was isolated from the treated cells with a QIAamp DNA mini kit (Qiagen). The DNA samples were diluted to 3 ng/μl for use in PCR reactions. PCR products were quantified using the Quant-iT Picogreen dsDNA Assay kit (Invitrogen). The relative amplification of the large band was normalized to untreated cells. The amplification of the small mitochondrial band was used to normalize the data obtained from the large band to account for mitochondrial DNA copy number.

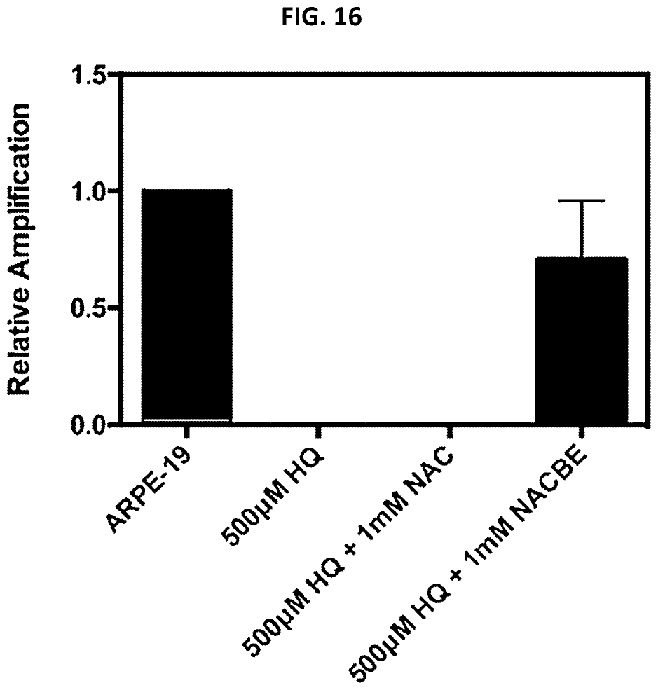

Results are shown in FIG. 16. Results showed that treatment with HQ drastically reduced the amplification of mitochondrial DNA, and that pretreatment with NAC did not show any signs of protection. However, pretreatment of cells with NACBE did keep the PCR amplification of the mitochondrial DNA intact.

1. A therapeutic composition comprising a therapeutic agent, wherein the therapeutic agent is an N-acetylcysteine (NAC) alkyl-ester analogue.

2. The therapeutic composition of claim 1, wherein the therapeutic agent is a NAC alkyl-ester analogue according to Formula (I): 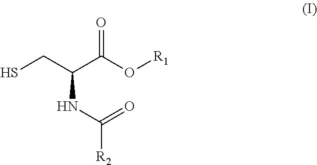
wherein R1 is a C1-C5 branched or linear alkyl group; and R2 is a C1-C3 alkyl group or a pyridyl group.

3. The therapeutic composition of claim 1, wherein the therapeutic agent is a NAC alkyl-ester analogue according to Formula (IA): 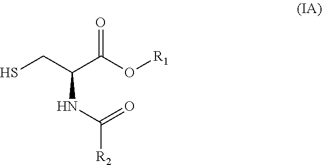
wherein R1 is a C1-C5 branched or linear alkyl group; and R2 is a C1-C2 alkyl group, or a pyridyl group.

4. The therapeutic composition of claim 2, wherein R1 is a C1-C4 linear alkyl group; and R2 is a C1-C2 alkyl group or C1-C3 alkyl group.

5. The therapeutic composition of claim 1, wherein the therapeutic agent is an N-acetylcysteine methyl ester (NACME).

6. The therapeutic composition of claim 1, wherein the therapeutic agent is an N-acetylcysteine ethyl ester (NACEE).

7. The therapeutic composition of claim 1, wherein the therapeutic agent is an N-acetylcysteine propyl ester (NACPE).

8. The therapeutic composition of claim 1, wherein the therapeutic agent is an N-acetylcysteine isopropyl ester.

9. The therapeutic composition of claim 1, wherein the therapeutic agent is an N-acetylcysteine butyl ester (NACBE).

10. The therapeutic composition of claim 2, wherein R1 is C1-C4 linear alkyl group; and R2 is a pyridyl group.

11. The therapeutic composition of claim 1, wherein therapeutic agent is an N-nicotinoylcysteine methyl ester (NNICME) or an N-nicotinoylcysteine ethyl ester (NNICEE) or an N-nicotinoylcysteine propyl ester (NNICEE).

12. An ocular implant comprising a biocompatible polymer and the therapeutic composition of claim 1.

13. The ocular implant of claim 12, wherein the biocompatible polymer comprises an ethylene-vinyl ester copolymer selected from: ethylene-vinyl acetate (EVA), ethylene-vinyl hexanoate (EVH), ethylene-vinyl propionate (EVP), ethylene-vinyl butyrate (EVB), ethylene vinyl pentantoate (EVP), ethylene-vinyl trimethyl acetate (EVTMA), ethylene-vinyl diethyl acetate (EVDEA), ethylene-vinyl 3-methylbutanoate (EVMB), ethylene-vinyl 3-3-dimethylbutanoate (EVDMB), ethylene-vinyl benzoate (EVBZ), or mixtures thereof.

14. (canceled)

15. The ocular implant of claim 1, wherein the implant is a multilayer implant comprising an outer layer and an inner layer; wherein the outer layer comprises a first polymer, and the outer layer comprises curvature at both an outer surface and an inner surface; and wherein the inner layer comprises the biocompatible polymer and the therapeutic composition, and the inner layer comprises curvature at both an outer surface and an inner surface; wherein the outer layer extends circumferentially beyond the inner layer such that the surface of the circumferential extension of the outer layer is capable of making contact with the sclera of an eye; and wherein at least one surface of the inner layer is capable of making contact with the sclera of the eye.

16-22. (canceled)

23. A method of treating an eye disorder in the eye of a subject, comprising:
(i) providing a therapeutic composition of claim 1; and
(ii) placing the therapeutic composition or the ocular implant into the sub-Tenon's space and in contact with the sclera of the eye of the subject.

24. The method of claim 23, wherein the eye disorder is macular degeneration.

25. The method of claim 23, wherein the eye disorder is age-related macular degeneration (AMD).

26. The method of claim 23, wherein the therapeutic composition is placed in the posterior of the eye near the macula of the eye.

27. The method of claim 23, wherein an applicator device is used to place the therapeutic composition into the sub-Tenon's space the eye.

28. A method of treating an eye disorder in the eye of a subject, comprising:
(i) providing an ocular implant of claim 12; and
(ii) placing the therapeutic composition or the ocular implant into the sub-Tenon's space and in contact with the sclera of the eye of the subject.
